(19) United States
(12) Patent Application Publication
(10) Pub. No.: US 2010/0249107 A1
(43) Pub. Date: Sep. 30, 2010

(54) Biomarkers for Alzheimer's Disease Progression

(75) Inventor: Jonathan Benjamin Singer, Somerville, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/438,457

(22) PCT Filed: Aug. 17, 2007
(86) PCT No.: PCT/US07/76159

§ 371 Date: Feb. 23, 2009

Related U.S. Application Data
(60) Provisional application No. 60/823,025, filed on Aug. 21, 2006.

(51) Int. Cl. A61K 31/55; C07C 271/40; A61K 31/27; A61K 31/435; A61K 31/4353; A61K 31/13; G01N 33/50; A61P 25/28

(52) U.S. Cl. 514/215; 560/163; 514/490; 514/297; 514/319; 514/662; 436/94; 436/86

(57) ABSTRACT

This invention relates generally to the analytical testing of tissue samples in vitro, and more particularly to aspects of genetic polymorphisms associated with the conversion from Mile Cognitive Impairment to dementia, e.g., Alzheimer's Disease (AD). The invention provides AD-associated mutations which are useful in the diagnosis, prognosis or therapeutic treatment of dementia, e.g., Alzheimer's Disease.

### FIELD OF THE INVENTION

This invention relates generally to the analytical testing of tissue samples in vitro, and more particularly to aspects of genetic polymorphisms useful in the diagnosis, prognosis or prevention/therapeutic treatment of dementia, e.g., Alzheimer's Disease.

### BACKGROUND OF THE INVENTION

Conventional medical approaches to diagnosis and treatment of disease are based on clinical data alone or made in conjunction with a diagnostic test. Such traditional practices often lead to therapeutic choices that are not optimal for the efficacy of the prescribed drug therapy or to minimize the likelihood of side effects for an individual subject. Therapy-specific diagnostics (a.k.a., theranostics) is an emerging medical technology field, which provides tests useful to diagnose a disease, choose the correct treatment regime and monitor a subject's response. That is, theranostics are useful to predict and assess drug response in individual subjects, i.e., individualized medicine. Theranostic tests are also useful to select subjects for treatments that are particularly likely to benefit from the treatment or to provide an early and objective indication of treatment efficacy in individual subjects, so that the treatment can be altered with a minimum of delay.

Progress in pharmacogenetics, which establishes correlations between responses to specific drugs and the genetic profile of individual patients, is foundational to the development of new theranostic approaches. As such, there is a need in the art for the evaluation of patient-to-patient variations in gene sequence and gene expression. A common form of genetic profiling relies on the identification of DNA sequence variations called single nucleotide polymorphisms (“SNPs”), which are one type of genetic mutation leading to patient-to-patient variation in individual drug response. It follows that there is a need in the art to identify and characterize genetic mutations, such as SNPs, which are useful to identify the genotypes of subjects associated with drug responsiveness, side-effects, or optimal dose.

Dementia is a brain disorder that seriously affects a person's ability to carry out daily activities. The most common form of dementia among older people is Alzheimer's disease (AD), which initially involves the parts of the brain that control thought, memory, and language. It is estimated that as many as 4.5 million Americans suffer from AD. This number is expected to quadruple by the year 2050 as the population ages. The disease usually begins after age 60, and risk increases with age. While younger people also may get AD, it is much less common. About 5 percent of men and women ages 65 to 74 have AD, and nearly half of those age 85 and older may have the disease. AD is not a normal part of aging. The cause(s) of AD are not completely known and there is no cure. The progression of AD symptoms may be temporarily slowed for people in the early and middle stages of the disease, with administration of drugs such as tacrine (Cognex), donepezil (Aricept), rivastigmine (Exelon), or galantamine (Razadyne, previously known as Reminyl).

Mild cognitive impairment (MCI) is a transition stage between the cognitive changes of normal aging and the more serious problems caused by Alzheimer's disease. MCI is different from both AD and normal age-related memory change. People with MCI have ongoing memory problems, but they do not experience other losses such as confusion, attention problems, and difficulty with language. MCI can progress, however, to Alzheimer's Disease. Accordingly, there is a need in the art to identify and characterize genetic mutations, which are useful to identify the genotypes of subjects associated with the onset and/or progression of dementia, e.g., Alzheimer's Disease.

### SUMMARY OF THE INVENTION

The present invention provides novel genetic mutations (i.e., “AD-associated mutations”), which are useful as biomarkers to identify the genotypes of subjects associated with the onset and/or progression of dementia, e.g., Alzheimer's Disease. The invention provides for the use of a Novartis compound, rivastigmine (Exelon), in the manufacture of a medicament for the treatment of Alzheimer's Disease with a reduced toxicity or increased effect in a selected patient population, wherein the patient population is selected on the basis of the presence of at least one gene mutation of the invention.

The invention also provides a method for treating Alzheimer's Disease in a subject. The genotype or haplotype of the subject is obtained at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11, so that the genotype and/or haplotype is indicative of a propensity of the Alzheimer's Disease to respond to the drug. Then, an anti-Alzheimer's Disease therapy is administered to the subject, including, but not limited to, e.g., tacrine; donepezil; rivastigmine; galantamine; or an AD-associated mutation modulating agent

The invention provides a method for identifying a subject with a disorder for which an Alzheimer's Disease-associated mutation of the invention is predictive. The genotype or haplotype of the subject is obtained at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11, and assessed to determine the presence of a mutation of the invention to identify the subject as having a propensity for having a disorder for which an Alzheimer's Disease-associated mutation is predictive. The disorder for which the Alzheimer's Disease-associated mutation is predictive can be Alzheimer's Disease.

The invention provides a method for determining, prior to initiation of treatment, which subject should be included in a study of a therapeutic or study agent by interrogating the genotype and/or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11 and then (i) including the subject in the study if the genotype is indicative of a propensity to Alzheimer's Disease by the subject; (ii) excluding the subject from the study if the genotype is not indicative of a propensity to Alzheimer's Disease by the subject; or (iii) both (i) and (ii).

The invention provides a method for determining the responsiveness of a subject with a disorder to treatment by obtaining the genotype or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11 and then assessing the genotype and/or haplotype to determine the presence of a mutation or polymorphism of the invention, wherein the presence of the mutation or polymorphism is indicative of an individual that is responsive to treatment of the disorder to identify the subject as responsive to treatment of the disorder.

The invention provides a method for determining, prior to treatment, which subject will develop toxicity when treated with a compound by obtaining the genotype or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF1 (i.e., “the genotypes or haplotypes of the invention”); and then assessing the genotype and/or haplotype to determine the presence of a mutation or polymorphism of the invention wherein the presence of the mutation or polymorphism is indicative of which subject will develop toxicity when treated in order to identify the subject as a subject that will develop toxicity when treated.

The invention further provides test kits for: (a) use in determining when a compound should be discontinued for an individual being treated with a therapeutic or study agent; (b) use in determining which individuals will develop toxicity; (c) for use in determining which individuals are developing toxicity when treated with a compound; and (d) for use in determining which individuals will develop toxicity when treated with a compound and should not be included in a study of that compound. The kits include a reagent for detecting a polynucleotide or polypeptide encoded by a gene having a sequence comprising a mutation of the invention, in a container suitable for contacting a body fluid with instructions for interpreting the results.

The invention provides a method for monitoring the progression or development of toxicity in a subject being treated with a compound, by: (a) providing a first test biological sample from the subject; (b) providing a second test biological sample from the subject which is later in time than the first test biological sample; (c) contacting the test biological samples with a reagent for detecting a polynucleotide or polypeptide encoded by a gene having a sequence comprising a mutation of the invention; (d) determining the level of expression of the polypeptide or polynucleotide in the test biological samples; and (e) comparing the level of the polynucleotide or polypeptide level in the first test biological sample with the level of the polynucleotide or polypeptide in the second test biological sample, wherein an increase or a decrease in the level of polynucleotide or polypeptide in the second test biological sample relative to the level of the polynucleotide or polypeptide in the first test biological sample indicates the progression or development of toxicity in the subject being treated with the compound.

The invention provides a method for determining when treatment with a compound should be discontinued in a subject at risk of having toxicity during or after treatment with the compound, by: (a) providing a test biological sample and a standard reference sample; (b) contacting the test biological sample with a reagent for detecting a polynucleotide or polypeptide encoded by a gene having a sequence comprising a mutation of the invention; (c) determining the level of expression of the polypeptide or polynucleotide in the test biological sample; and (d) comparing the level of the polynucleotide or polypeptide level in the test biological sample with the level of the polynucleotide or polypeptide in a standard reference sample, wherein similarity between the level of polynucleotide or polypeptide and the level of the polynucleotide or polypeptide in the standard reference sample is indicative of the development of toxicity in the subject and determines that the compound should be discontinued.

The invention provides a method for validating a compound as a candidate target for treating a medical condition predicted to be associated with Alzheimer's Disease or MCI by: (a) comparing the frequency of each of the genotypes or haplotypes of the invention between first and second populations, wherein the first population is a group of individuals having the medical condition and the second population is a group of individuals lacking the medical condition; and (b) making a decision whether to pursue the compound for treating the medical condition; wherein if at least one of the haplotypes is present in a frequency in the first population that is different from the frequency in the second population at a statistically significant level, then the decision is to pursue the compound and if none of the haplotypes are seen in a different frequency, at a statistically significant level, between the first and second populations, then the decision is to not pursue the compound.

The invention also provides a computer system for storing and analyzing polymorphism data for a gene with: (a) a central processing unit (CPU); (b) a communication interface; (c) a display device; (d) an input device; and (e) a database containing the polymorphism data, wherein the polymorphism data comprises the haplotypes of the invention.

### DETAILED DESCRIPTION OF THE INVENTION

Mild cognitive impairment, or MCI, affects many areas of cognition and can be divided into two broad subtypes. One subtype, amnestic MCI, significantly affects memory. The other type, nonamnestic MCI, does not. Other functions, such as language, attention and visuospatial skills, may be impaired in either type. The exact prevalence of MCI has been estimated as high as 20 percent of the nondemented population over age 65. About a third of those cases, however, have the amnestic variety that has been linked to Alzheimer's. Amnestic (memory-related) MCI converts to Alzheimer's at a rate of 10 percent to 15 percent a year.

The present invention provides novel genetic mutations (i.e., “AD-associated mutations”), which are useful to identify the genotypes of subjects associated with the onset and/or progression of dementia, e.g., Alzheimer's Disease. Specifically, 420 patient samples from the InDDex (ENA713B IA07) clinical trial were genotyped using the Affymetrix 100K genotyping platform and whole-genome association analysis was performed, on single points and inferred haplotype blocks, using the case-control test of Haploview (Barrett et al., Bioinformatics 21:263-265 (2005)). (See generally EXAMPLE I). The outcome phenotype was conversion or non-conversion from MCI to AD.

The invention provides twenty-five (25) AD-associated mutations (e.g., MUT-1 through MUT-25) which are summarized below in TABLE 1. The AD-associated mutations of Table 1 have not previously been associated with AD, although several of them are in pathways that have been linked to the disease. The AD-associated mutations of the present invention are useful in the diagnosis, prognosis or therapeutic treatment of dementia, e.g., Alzheimer's Disease. In one aspect, the AD-associated mutations of the invention are useful as diagnostic/predictive markers of AD. In another aspect, the AD-associated mutations of the invention are useful as targets for agents useful in the prevention or treatment of AD.

TABLE 1
AD-Associated Mutations of the Invention

| | | | Allele | Allele | dbSNP | Biomarker |
|---|---|---|---|---|---|---|
| Name | Chrom | Position | A | B | RS ID | Gene |
| MUT-1 | 2 | 214935064 | G | T | rs10490502 | SPAG16 |
| (SNP_A-1670809) | | | | | | |
| MUT-2 | 2 | 124721329 | C | T | rs1170585 | CNTNAP5 |
| (SNP_A-1750208) | | | | | | |
| MUT-3 | 5 | 153057231 | A | G | rs4415128 | GRIA1 |
| (SNP_A-1747985) | | | | | | |
| MUT-4 | 7 | 86116564 | C | T | rs17126 | — |
| (SNP_A-1672398) | | | | | | |
| MUT-5 | 5 | 36703336 | C | G | rs10491374 | SLC1A3 |
| (SNP_A-1654099) | | | | | | |
| MUT-6 | 18 | 60211031 | A | T | rs1833486 | C18orf20 |
| (SNP_A-1653812) | | | | | | |
| MUT-7 | 5 | 153177889 | A | T | rs2964013 | GRIA1 |
| (SNP_A-1708263) | | | | | | C5orf3 |
| MUT-8 | 13 | 41916177 | A | T | rs720824 | TNFSF11 |
| (SNP_A-1646220) | | | | | | |
| MUT-9 | 5 | 153067976 | C | T | rs7735784 | GRIA1 |
| (SNP_A-1671292) | | | | | | |
| MUT-10 | 1 | 43906558 | A | G | rs7516647 | ST3GAL3 |
| (SNP_A-1754998) | | | | | | |
| MUT-11 | 7 | 16181205 | G | T | rs1921840 | — |
| (SNP_A-1744334) | | | | | | |
| MUT-12 | 6 | 66726185 | C | T | rs2061578 | BAI3 |
| (SNP_A-1694932) | | | | | | |
| MUT-13 | 7 | 86115205 | A | C | rs6465088 | — |
| (SNP_A-1677855) | | | | | | |
| MUT-14 | 8 | 129710034 | C | T | rs10505528 | MGC27434 |
| (SNP_A-1659634) | | | | | | |
| MUT-15 | 7 | 81502544 | G | T | rs929351 | CACNA2D1 |
| (SNP_A-1657336) | | | | | | |
| MUT-16 | 9 | 27057529 | C | T | rs10511798 | LRRC19 |
| (SNP_A-1742608) | | | | | | TEK CCDC2 |
| MUT-17 | 3 | 103365250 | A | G | rs9290621 | NFKBIZ |
| (SNP_A-1681449) | | | | | | LOC131368 |
| MUT-18 | 18 | 60165056 | C | T | rs4306624 | C18orf20 |
| (SNP_A-1674524) | | | | | | |
| MUT-19 | 8 | 56479295 | C | G | rs2622542 | — |
| (SNP_A-1689621) | | | | | | |
| MUT-20 | 1 | 77566211 | A | T | rs10518551 | AK5 |
| (SNP_A-1728960) | | | | | | |
| MUT-21 | 15 | 31572775 | A | G | rs1369308 | — |
| (SNP_A-1666270) | | | | | | |
| MUT-22 | 18 | 19749481 | C | T | rs1541836 | LAMA3 |
| (SNP_A-1738563) | | | | | | |
| MUT-23 | 3 | 109243490 | C | T | rs696365 | CD47 |
| (SNP_A-1748758) | | | | | | BBX |
| MUT-24 | 18 | 35487281 | A | G | rs10502726 | PIK3C3 |
| (SNP_A-1652367) | | | | | | |
| MUT-25 | 3 | 109276399 | C | T | rs3804640 | CD47 |
| (SNP_A-1752416) | | | | | | |

It is to be appreciated that certain aspects, modes, embodiments, variations and features of the invention are described below in various levels of detail in order to provide a substantial understanding of the present invention. In general, such disclosure provides new AD-associated mutations, including SNPs, useful in the diagnosis and treatment of subjects in need thereof. Accordingly, the various aspects of the present invention relate to polynucleotides encoding the AD-associated mutations of the invention, expression vectors encoding the AD-associated mutant polypeptides of the invention and organisms that express the AD-associated mutant polynucleotides and/or AD-associated mutant polypeptides of the invention. The various aspects of the present invention further relate to diagnostic/theranostic methods and kits that use the AD-associated mutations of the invention to identify individuals predisposed to disease or to classify individuals with regard to drug responsiveness, side effects, or optimal drug dose. In other aspects, the invention provides methods for compound validation and a computer system for storing and analyzing data related to the AD-associated mutations of the invention. Accordingly, various particular embodiments that illustrate these aspects follow.

Definitions. The definitions of certain terms as used in this specification are provided below. Definitions of other terms may be found in the glossary provided by the U.S. Department of Energy, Office of Science, Human Genome Project (http://www.ornl.gov/sci/techresources/Human_Genome/glossary/). In practicing the present invention, many conventional techniques in molecular biology, microbiology and recombinant DNA are used. These techniques are well-known and are explained in, e.g., Current Protocols in Molecular Biology, Vols. I-III, Ausubel, ed. (1997); Sambrook et al., Molecular Cloning: A Laboratory Manual, Second Edition (Cold Spring Harbor Laboratory Press, Cold Spring Harbor, N.Y., 1989); DNA Cloning: A Practical Approach, Vols. I and II, Glover D, ed. (1985); Oligonucleotide Synthesis, Gait, ed. (1984); Nucleic Acid Hybridization, Hames & Higgins, Eds. (1985); Transcription and Translation, Hames & Higgins, eds. (1984); Animal Cell Culture, Freshney, ed. (1986); Immobilized Cells and Enzymes (IRL Press, 1986); Perbal, A Practical Guide to Molecular Cloning; the series, Methods in Enzymol. (Academic Press, Inc., 1984); Gene Transfer Vectors for Mammalian Cells, Miller and Calos, Eds. (Cold Spring Harbor Laboratory, NY, 1987); and Methods in Enzymology, Vols. 154 and 155, Wu and Grossman, and Wu, Eds., respectively.

As used herein, the term “allele” means a particular form of a gene or DNA sequence at a specific chromosomal location (locus).

As used herein, the term “antibody” includes, but is not limited to, polyclonal antibodies, monoclonal antibodies, humanized or chimeric antibodies and biologically functional antibody fragments sufficient for binding of the antibody fragment to the protein.

As used herein, the term “clinical response” means any or all of the following: a quantitative measure of the response, no response, and adverse response (i.e., side effects).

As used herein, the term “clinical trial” means any research study designed to collect clinical data on responses to a particular treatment, and includes but is not limited to phase I, phase II and phase III clinical trials. Standard methods are used to define the patient population and to enrol subjects.

As used herein, the term “effective amount” of a compound is a quantity sufficient to achieve a desired therapeutic and/or prophylactic effect, for example, an amount which results in the prevention of or a decrease in the symptoms associated with a disease that is being treated, e.g., the diseases associated with AD-associated mutant polynucleotides and mutant polypeptides identified herein. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pairs found at one or more polymorphic sites in a locus on a pair of homologous chromosomes in an individual. As used herein, genotype includes a full-genotype and/or a sub-genotype.

As used herein, the term “locus” means a location on a chromosome or DNA molecule corresponding to a gene or a physical or phenotypic feature.

As used herein, the term “AD-associated mutation modulating agent” is any compound that alters (e.g., increases or decreases) the expression level or biological activity level of AD-associated mutant polypeptide compared to the expression level or biological activity level of AD-associated mutant polypeptide in the absence of the AD-associated modulating agent. AD-associated mutation modulating agent can be a small molecule, polypeptide, carbohydrate, lipid, nucleotide, or combination thereof. The AD-associated mutation modulating agent may be an organic compound or an inorganic compound.

As used herein, the term “mutant” means any heritable variation from the wild-type that is the result of a mutation, e.g., single nucleotide polymorphism (“SNP”). The term “mutant” is used interchangeably with the terms “marker”, “biomarker”, and “target” throughout the specification.

As used herein, the term “medical condition” includes, but is not limited to, any condition or disease manifested as one or more physical and/or psychological symptoms for which treatment is desirable, and includes previously and newly identified diseases and other disorders.

As used herein, the term “nucleotide pair” means the nucleotides found at a polymorphic site on the two copies of a chromosome from an individual.

As used herein, the term “polymorphic site” means a position within a locus at which at least two alternative sequences are found in a population, the most frequent of which has a frequency of no more than 99%.

As used herein, the term “phased” means, when applied to a sequence of nucleotide pairs for two or more polymorphic sites in a locus, the combination of nucleotides present at those polymorphic sites on a single copy of the locus is known.

As used herein, the term “polymorphism” means a difference in DNA sequence among individuals, or genetic variations. Genetic variations occurring in more than 1% of a population are considered useful polymorphisms for genetic linkage analysis. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “single nucleotide polymorphism (SNP)” means nucleotide variability at a single position in the genome, in which two alternative bases occur at appreciable frequency (i.e., >1%) in the human population. A SNP may occur within a gene or within intergenic regions of the genome. SNP probes according to the invention are oligonucleotides that are complementary to a SNP and its flanking nucleic acid sequence(s).

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., cynomolgus monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

Predictive Medicine. In one aspect, the present invention pertains to the field of predictive medicine in which diagnostic assays, prognostic assays, pharmacogenetics and monitoring clinical trials are used for prognostic (predictive) purposes to thereby diagnose and treat a subject prophylactically. Accordingly, one aspect of the present invention relates to diagnostic assays for determining biomarker protein and/or nucleic acid expression (e.g., MUT-1 through MUT-25 protein or nucleic acid) from a sample (e.g., blood, serum, cells, tissue) to thereby determine whether a subject is likely to convert from MCI to AD.

Another aspect of the invention pertains to monitoring the influence of agents (e.g., drugs, compounds) on the expression or activity of biomarker in clinical trials as described in further detail in the following sections.

An exemplary method for detecting the presence or absence of biomarker proteins or genes of the invention in a sample involves obtaining a sample from a test subject and contacting the sample with a compound or an agent capable of detecting the protein or nucleic acid (e.g., mRNA, genomic DNA) that encodes the biomarker protein such that the presence of the biomarker protein or nucleic acid is detected in the sample. A preferred agent for detecting mRNA or genomic DNA corresponding to a biomarker gene or protein of the invention is a labelled nucleic acid probe capable of hybridizing to an mRNA or genomic DNA of the invention. Suitable probes for use in the diagnostic assays of the invention are described herein.

A preferred agent for detecting biomarker protein is an antibody capable of binding to biomarker protein, preferably an antibody with a detectable label. Antibodies can be polyclonal, or more preferably, monoclonal. An intact antibody, or a fragment thereof (e.g., Fab or F(ab′)₂) can be used. The term “labelled”, with regard to the probe or antibody, is intended to encompass direct labelling of the probe or antibody by coupling (i.e., physically linking) a detectable substance to the probe or antibody, as well as indirect labelling of the probe or antibody by reactivity with another reagent that is directly labelled. Examples of indirect labelling include detection of a primary antibody using a fluorescently labelled secondary antibody and end-labelling of a DNA probe with biotin such that it can be detected with fluorescently labelled streptavidin.

The term “sample” is intended to include tissues, cells and biological fluids isolated from a subject, as well as tissues, cells and fluids present within a subject. That is, the detection method of the invention can be used to detect biomarker mRNA, protein, or genomic DNA in a sample in vitro as well as in vivo. For example, in vitro techniques for detection of biomarker mRNA include Northern hybridizations and in situ hybridizations. In vitro techniques for detection of biomarker protein include enzyme linked immunosorbent assays (ELISAs), Western blots, immunoprecipitations and immunofluorescence. In vitro techniques for detection of biomarker genomic DNA include Southern hybridizations. Furthermore, in vivo techniques for detection of biomarker protein include introducing, into a subject, a labelled anti-biomarker antibody. For example, the antibody can be labelled with a radioactive biomarker whose presence and location in a subject can be detected by standard imaging techniques.

In one embodiment, the sample contains protein molecules from the test subject. Alternatively, the sample can contain mRNA molecules from the test subject or genomic DNA molecules from the test subject. A preferred sample is a serum sample isolated by conventional means from a subject.

The methods further involve obtaining a control sample (e.g., sample from a non-demented subject showing no sign of AD) from a control subject, contacting the control sample with a compound or agent capable of detecting biomarker protein, mRNA, or genomic DNA, such that the presence of biomarker protein, mRNA or genomic DNA is detected in the sample, and comparing the presence of biomarker protein, mRNA or genomic DNA in the control sample with the presence of biomarker protein, mRNA or genomic DNA in the test sample.

The diagnostic methods described herein can furthermore be utilized to identify subjects having or at risk of developing a disease or disorder associated with aberrant biomarker expression or activity (i.e., having one or more of the AD-associated mutations/genes of the invention). As used herein, the term “aberrant” includes a biomarker expression or activity which deviates from the wild type biomarker expression or activity. Aberrant expression or activity includes increased or decreased expression or activity, as well as expression or activity which does not follow the wild-type developmental pattern of expression or the subcellular pattern of expression. For example, aberrant biomarker expression or activity is intended to include the cases in which a mutation in the biomarker gene causes the biomarker gene to be under-expressed or over-expressed and situations in which such mutations result in a non-functional biomarker protein or a protein which does not function in a wild-type fashion, e.g., a protein which does not interact with a biomarker ligand or one which interacts with a non-biomarker protein ligand.

Furthermore, the prognostic assays described herein can be used to determine whether a subject can be administered an agent (e.g., an agonist, antagonist, peptidomimetic, protein, peptide, nucleic acid, small molecule, or other drug candidate) to reduce the risk of the onset or progression of dementia, e.g., AD. Thus, the present invention provides methods for determining whether a subject can be effectively treated with an agent for a disorder associated with increased or decreased gene expression or activity of one or more of the AD-associated mutations/genes of the invention.

Monitoring the influence of agents (e.g., drugs) on the expression or activity of genes can be applied not only in basic drug screening, but also in clinical trials. For example, the effectiveness of an agent determined by a screening assay as described herein to modulate (i.e., increase or decrease) biomarker gene expression, protein levels, or up-regulate activity, can be monitored in clinical trials of subjects exhibiting Alzheimer's Disease or MCI-related symptoms by examining the molecular signature and any changes in the molecular signature during treatment with an agent.

For example, and not by way of limitation, genes and their encoded proteins that are modulated in cells by treatment with an agent (e.g., compound, drug or small molecule) which modulates gene activity can be identified. In a clinical trial, cells can be isolated and RNA prepared and analyzed for the levels of expression of genes implicated associated with dementia. The levels of gene expression (e.g., a gene expression pattern) can be quantified by northern blot analysis or RT-PCR, as described herein, or alternatively by measuring the amount of protein produced, by one of the methods as described herein. In this way, the gene expression pattern can serve as a molecular signature, indicative of the physiological response of the cells to the agent. Accordingly, this response state may be determined before, and at various points during treatment of the subject with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a gene or combination of genes, the protein encoded by the genes, mRNA, or genomic DNA in the preadministration sample; (iii) obtaining one or more post-administration samples from the subject; (iv) detecting the level of expression or activity of the biomarker protein, mRNA, or genomic DNA in the post-administration samples; (v) comparing the level of expression or activity of the biomarker protein, mRNA, or genomic DNA in the pre-administration sample with the a gene or combination of genes, the protein encoded by the genes, mRNA, or genomic DNA in the post administration sample or samples; and (vi) altering the administration of the agent to the subject accordingly. For example, increased administration of the agent may be desirable to decrease the expression or activity of the genes to lower levels, i.e., to increase the effectiveness of the agent to protect against the onset or progression of AD. Alternatively, decreased administration of the agent may be desirable to decrease expression or activity of biomarker to lower levels than detected, i.e., to decrease the effectiveness of the agent e.g., to avoid toxicity. According to such an embodiment, gene expression or activity may be used as an indicator of the effectiveness of an agent, even in the absence of an observable phenotypic response.

Identification and Characterization of Gene Sequence Variation. Due to their prevalence and widespread nature, SNPs have the potential to be important tools for locating genes that are involved in human disease conditions. See e.g., Wang et al., Science 280: 1077-1082 (1998). It is increasingly clear that the risk of developing many common disorders and the metabolism of medications used to treat these conditions are substantially influenced by underlying genomic variations, although the effects of any one variant might be small.

A SNP is said to be “allelic” in that due to the existence of the polymorphism, some members of a species may have an unmutated sequence (i.e., the original allele) whereas other members may have a mutated sequence (i.e., the variant or mutant allele).

An association between a SNP and a particular phenotype does not necessarily indicate or require that the SNP is causative of the phenotype. Instead, the association may merely be due to genome proximity between a SNP and those genetic factors actually responsible for a given phenotype, such that the SNP and said genetic factors are closely linked. That is, a SNP may be in linkage disequilibrium (“LD”) with the “true” functional variant. LD (a.k.a., allelic association) exists when alleles at two distinct locations of the genome are more highly associated than expected. Thus, a SNP may serve as a marker that has value by virtue of its proximity to a mutation that causes a particular phenotype.

In describing the polymorphic sites of the invention, reference is made to the sense strand of the gene for convenience. As recognized by the skilled artisan, however, nucleic acid molecules containing the gene may be complementary double stranded molecules and thus reference to a particular site on the sense strand refers as well to the corresponding site on the complementary antisense strand. That is, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the genomic variants described herein.

Identification and Characterization of SNPs. Many different techniques can be used to identify and characterize SNPs, including single-strand conformation polymorphism (SSCP) analysis, heteroduplex analysis by denaturing high-performance liquid chromatography (DHPLC) and direct DNA sequencing and computational methods. Shi et al., Clin. Chem. 47:164-172 (2001). There is a wealth of sequence information in public databases.

The most common SNP-typing methods currently include hybridization, primer extension, and cleavage methods. Each of these methods must be connected to an appropriate detection system. Detection technologies include fluorescent polarization (Chan et al., Genome Res. 9:492-499 (1999)), luminometric detection of pyrophosphate release (pyrosequencing) (Ahmadiian et al., Anal. Biochem. 280:103-10 (2000)), fluorescence resonance energy transfer (FRET)-based cleavage assays, DHPLC, and mass spectrometry (Shi, Clin. Chem. 47:164-172 (2001); U.S. Pat. No. 6,300,076 B1). Other methods of detecting and characterizing SNPs are those disclosed in U.S. Pat. Nos. 6,297,018 and 6,300,063.

Polymorphisms can also be detected using commercially available products, such as INVADER™ technology (available from Third Wave Technologies Inc. Madison, Wis., USA). In this assay, a specific upstream “invader” oligonucleotide and a partially overlapping downstream probe together form a specific structure when bound to complementary DNA template. This structure is recognized and cut at a specific site by the Cleavase enzyme, resulting in the release of the 5′ flap of the probe oligonucleotide. This fragment then serves as the “invader” oligonucleotide with respect to synthetic secondary targets and secondary fluorescently labelled signal probes contained in the reaction mixture. See also, Ryan D et al., Molecular Diagnosis 4(2): 135-144 (1999) and Lyamichev V et al., Nature Biotechnology 17: 292-296 (1999), see also U.S. Pat. Nos. 5,846,717 and 6,001,567.

The identity of polymorphisms may also be determined using a mismatch detection technique including, but not limited to, the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich P, Ann Rev Genet. 25:229-253 (1991)). Alternatively, variant alleles can be identified by single strand conformation polymorphism (SSCP) analysis (Orita et al., Genomics 5:874-879 (1989); Humphries et al., in Molecular Diagnosis of Genetic Diseases, R. Elles, ed., pp. 321-340 (1996)) or denaturing gradient gel electrophoresis (DGGE) (Wartell et al., Nucl. Acids. Res. 18:2699-2706 (1990); Sheffield et al., Proc. Natl. Acad. Sci. USA 86:232-236 (1989)). A polymerase-mediated primer extension method may also be used to identify the polymorphisms. Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, and U.S. Pat. Nos. 5,302,509 and 5,945,283. Extended primers containing a polymorphism may be detected by mass spectrometry as described in U.S. Pat. No. 5,605,798. Another primer extension method is allele-specific PCR (Ruafio et al., Nucl. Acids. Res. 17:8392 (1989); Ruafio et al., Nucl. Acids. Res. 19: 6877-6882 (1991); WO 93/22456; Turki et al., J. Clin. Invest. 95:1635-1641 (1995)). In addition, multiple polymorphic sites may be investigated by simultaneously amplifying multiple regions of the nucleic acid using sets of allele-specific primers as described in PCT patent application WO 89/10414.

Haplotyping and Genotyping Oligonucleotides. The invention provides methods and compositions for haplotyping and/or genotyping the gene in an individual. As used herein, the terms “genotype” and “haplotype” mean the genotype or haplotype containing the nucleotide pair or nucleotide, respectively, that is present at one or more of the polymorphic sites described herein and may optionally also include the nucleotide pair or nucleotide present at one or more additional polymorphic sites in the gene. The additional polymorphic sites may be currently known polymorphic sites or sites that are subsequently discovered.

The compositions of the invention contain oligonucleotide probes and primers designed to specifically hybridize to one or more target regions containing, or that are adjacent to, a polymorphic site. Oligonucleotide compositions of the invention are useful in methods for genotyping and/or haplotyping a gene in an individual. The methods and compositions for establishing the genotype or haplotype of an individual at the polymorphic sites described herein are useful for studying the effect of the polymorphisms in the aetiology of diseases affected by the expression and function of the protein, studying the efficacy of drugs targeting, predicting individual susceptibility to diseases affected by the expression and function of the protein and predicting individual responsiveness to drugs targeting the gene product.

Genotyping oligonucleotides of the invention may be immobilized on or synthesized on a solid surface such as a microchip, bead, or glass slide. See, e.g., WO 98/20020 and WO 98/20019.

Genotyping oligonucleotides may hybridize to a target region located one to several nucleotides downstream of one of the polymorphic sites identified herein. Such oligonucleotides are useful in polymerase-mediated primer extension methods for detecting one of the polymorphisms described herein and therefore such genotyping oligonucleotides are referred to herein as “primer-extension oligonucleotides”.

Direct Genotyping Method of the Invention. A genotyping method of the invention may involve isolating from an individual a nucleic acid mixture comprising the two copies of a gene of interest or fragment thereof, and determining the identity of the nucleotide pair at one or more of the polymorphic sites in the two copies. As will be readily understood by the skilled artisan, the two “copies” of a gene in an individual may be the same allele or may be different alleles. In a particularly preferred embodiment, the genotyping method comprises determining the identity of the nucleotide pair at each polymorphic site. Typically, the nucleic acid mixture is isolated from a biological sample taken from the individual, such as a blood sample or tissue sample. Suitable tissue samples include whole blood, semen, saliva, tears, urine, faecal material, sweat, buccal smears, skin and hair.

Direct Haplotyping Method of the Invention. A haplotyping method of the invention may include isolating from an individual a nucleic acid molecule containing only one of the two copies of a gene of interest, or a fragment thereof, and determining the identity of the nucleotide at one or more of the polymorphic sites in that copy. Direct haplotyping methods include, for example, CLASPER System™ technology (U.S. Pat. No. 5,866,404) or allele-specific long-range PCR (Michalotos-Beloin et al., Nucl. Acids. Res. 24: 4841-4843 (1996)). The nucleic acid may be isolated using any method capable of separating the two copies of the gene or fragment. As will be readily appreciated by those skilled in the art, any individual clone will only provide haplotype information on one of the two gene copies present in an individual. In one embodiment, a haplotype pair is determined for an individual by identifying the phased sequence of nucleotides at one or more of the polymorphic sites in each copy of the gene that is present in the individual. In a preferred embodiment, the haplotyping method comprises identifying the phased sequence of nucleotides at each polymorphic site in each copy of the gene.

In both the genotyping and haplotyping methods, the identity of a nucleotide (or nucleotide pair) at a polymorphic site may be determined by amplifying a target regions containing the polymorphic sites directly from one or both copies of the gene, or fragments thereof, and sequencing the amplified regions by conventional methods. The genotype or haplotype for the gene of an individual may also be determined by hybridization of a nucleic sample containing one or both copies of the gene to nucleic acid arrays and subarrays such as described in WO 95/11995.

Indirect Genotyping Method using Polymorphic Sites in Linkage Disequilibrium with a Target Polymorphism. In addition, the identity of the alleles present at any of the polymorphic sites of the invention may be indirectly determined by genotyping other polymorphic sites in linkage disequilibrium with those sites of interest. As described above, two sites are said to be in linkage disequilibrium if the presence of a particular variant at one site is indicative of the presence of another variant at a second site. Stevens J C, Mol. Diag. 4: 309-317 (1999). Polymorphic sites in linkage disequilibrium with the polymorphic sites of the invention may be located in regions of the same gene or in other genomic regions.

Amplifying a Target Gene Region. The target regions may be amplified using any oligonucleotide-directed amplification method, including but not limited to polymerase chain reaction (PCR). (U.S. Pat. No. 4,965,188), ligase chain reaction (LCR) (Barany et al., Proc. Natl. Acad. Sci. USA 88:189-193 (1991); published PCT patent application WO 90/01069), and oligonucleotide ligation assay (OLA) (Landegren et al., Science 241: 1077-1080 (1988)). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, published PCT patent application WO 89/06700) and isothermal methods (Walker et al., Proc. Natl. Acad. Sci. USA 89:392-396 (1992)).

Hybridizing Allele-Specific Oligonucleotide to a Target Gene. A polymorphism in the target region may be assayed before or after amplification using one of several hybridization-based methods known in the art. Typically, allele-specific oligonucleotides are utilized in performing such methods. The allele-specific oligonucleotides may be used as differently labelled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking, baking, etc. Allele-specific oligonucleotide may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibres, chips, dishes, and beads. The solid support may be treated, coated or derivatised to facilitate the immobilization of the allele-specific oligonucleotide or target nucleic acid.

Determining Population Genotypes and Haplotypes and Correlating Them with a Trait. The invention provides a method for determining the frequency of a genotype or haplotype in a population. The method comprises determining the genotype or the haplotype for a gene present in each member of the population, wherein the genotype or haplotype comprises the nucleotide pair or nucleotide detected at one or more of the polymorphic sites in the gene, and calculating the frequency at which the genotype or haplotype is found in the population. The population may be a reference population, a family population, a same sex population, a population group, or a trait population (e.g., a group of individuals exhibiting a trait of interest such as a medical condition or response to a therapeutic treatment).

In another aspect of the invention, frequency data for genotypes and/or haplotypes found in a reference population are used in a method for identifying an association between a trait and a genotype or a haplotype. The trait may be any detectable phenotype, including but not limited to susceptibility to a disease or response to a treatment. The method involves obtaining data on the frequency of the genotypes or haplotypes of interest in a reference population and comparing the data to the frequency of the genotypes or haplotypes in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

The frequency data for the reference and/or trait populations are obtained by accessing previously determined frequency data, which may be in written or electronic form. For example, the frequency data may be present in a database that is accessible by a computer. Once the frequency data are obtained, the frequencies of the genotypes or haplotypes of interest in the reference and trait populations are compared.

When polymorphisms are being analyzed, a calculation may be performed to correct for a significant association that might be found by chance. For statistical methods useful in the methods of the invention, see Statistical Methods in Biology, 3^{rd} edition, Bailey N T J, (Cambridge Univ. Press, 1997); Waterman M S, Introduction to Computational Biology (CRC Press, 2000) and Bioinformatics, Baxevanis A D & Ouellette B F F editors (John Wiley & Sons, Inc., 2001).

In another embodiment, the haplotype frequency data for different groups are examined to determine whether they are consistent with Hardy-Weinberg equilibrium. D. L. Hartl et al., Principles of Population Genomics, 3 rd Ed. (Sinauer Associates, Sunderland, Mass., 1997).

In another embodiment, statistical analysis is performed by the use of standard ANOVA tests with a Bonferroni correction or a bootstrapping method that simulates the genotype phenotype correlation many times and calculates a significance value. ANOVA is used to test hypotheses about whether a response variable is caused by or correlates with one or more traits or variables that can be measured. L D Fisher & G vanBelle, Biostatistics: A Methodology for the Health Sciences, Ch. 10 (Wiley-Interscience, New York, 1993).

In one embodiment for predicting a haplotype pair, the analysis includes an assigning step, as follows: First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair.

In another embodiment, a detectable genotype or haplotype that is in linkage disequilibrium with a genotype or haplotype of interest may be used as a surrogate marker. A genotype that is in linkage disequilibrium with another genotype is indicated where a particular genotype or haplotype for a given gene is more frequent in the population that also demonstrates the potential surrogate marker genotype than in the reference population. If the frequency is statistically significant, then the marker genotype is predictive of that genotype or haplotype, and can be used as a surrogate marker.

Another method for finding correlations between haplotype content and clinical responses uses predictive models based on error-minimizing optimization algorithms, one of which is a genetic algorithm. See, R Judson, “Genetic Algorithms and Their Uses in Chemistry” in Reviews in Computational Chemistry, Ch. 10, K B Lipkowitz & D B Boyd, eds. (VCH Publishers, New York, 1997) pp. 1-73. Simulated annealing (Press et al., Numerical Recipes in C: The Art of Scientific Computing, Ch. 10 (Cambridge University Press, Cambridge, 1992), neural networks (E Rich & K Knight, Artificial Intelligence, 2nd Edition, Ch. 10 (McGraw-Hill, New York, 1991), standard gradient descent methods (Press et al., supra Ch. 10), or other global or local optimization approaches (see discussion in Judson, supra) can also be used.

Correlating Subject Genotype or Haplotype to Treatment Response. In preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome, including efficacy measurements, pharmacokinetic measurements and side-effect measurements.

In another preferred embodiment, the trait of interest is a clinical response exhibited by a patient to some therapeutic treatment, for example, response to a drug targeting or to a therapeutic treatment for a medical condition.

To deduce a correlation between a clinical response to a treatment and a genotype or haplotype, genotype or haplotype data is obtained on the clinical responses exhibited by a population of individuals who received the treatment, hereinafter the “clinical population”. This clinical data may be obtained by analyzing the results of a clinical trial that has already been run and/or by designing and carrying out one or more new clinical trials.

The individuals included in the clinical population are usually graded for the existence of the medical condition of interest. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population, and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the gene for each individual in the trial population is genotyped and/or haplotyped, which may be done before or after administering the treatment.

These results are then analyzed to determine if any observed variation in clinical response between polymorphism groups is statistically significant. Statistical analysis methods, which may be used, are described in L. D. Fisher & G. vanBelle, Biostatistics: A Methodology for the Health Sciences (Wiley-Interscience, New York, 1993). This analysis may also include a regression calculation of which polymorphic sites in the gene contribute most significantly to the differences in phenotype.

After both the clinical and polymorphism data have been obtained, correlations between individual response and genotype or haplotype content are created. Correlations may be produced in several ways. In one method, individuals are grouped by their genotype or haplotype (or haplotype pair) (also referred to as a polymorphism group), and then the averages and standard deviations of clinical responses exhibited by the members of each polymorphism group are calculated.

From the analyses described above, the skilled artisan that predicts clinical response as a function of genotype or haplotype content may readily construct a mathematical model. The identification of an association between a clinical response and a genotype or haplotype (or haplotype pair) for the gene may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic sites in the gene), a serological test, or a physical exam measurement. The only requirement is that there be a good correlation between the diagnostic test results and the underlying genotype or haplotype. In a preferred embodiment, this diagnostic method uses the predictive haplotyping method described above.

Assigning a Subject to a Genotype Group. As one of skill in the art will understand, there will be a certain degree of uncertainty involved in making this determination. Therefore, the standard deviations of the control group levels would be used to make a probabilistic determination and the methods of this invention would be applicable over a wide range of probability based genotype group determinations. Thus, for example and not by way of limitation, in one embodiment, if the measured level of the gene expression product falls within 2.5 standard deviations of the mean of any of the control groups, then that individual may be assigned to that genotype group. In another embodiment if the measured level of the gene expression product falls within 2.0 standard deviations of the mean of any of the control groups then that individual may be assigned to that genotype group. In still another embodiment, if the measured level of the gene expression product falls within 1.5 standard deviations of the mean of any of the control groups then that individual may be assigned to that genotype group. In yet another embodiment, if the measured level of the gene expression product is 1.0 or less standard deviations of the mean of any of the control groups levels then that individual may be assigned to that genotype group.

Thus this process allows determination, with various degrees of probability, which group a specific subject should be placed in, and such assignment to a genotype group would then determine the risk category into which the individual should be placed.

Correlation between Clinical Response and Genotype or Haplotype. In order to deduce a correlation between clinical response to a treatment and a genotype or haplotype, it is necessary to obtain data on the clinical responses exhibited by a population of individuals who received the treatment, hereinafter the “clinical population.” This clinical data may be obtained by analyzing the results of a clinical trial that has already been run and/or the clinical data may be obtained by designing and carrying out one or more new clinical trials.

The standard control levels of the gene expression product, thus determined in the different genotype or haplotype groups, would then be compared with the measured level of a gene expression product in a given patient. This gene expression product could be the characteristic mRNA associated with that particular genotype group or the polypeptide gene expression product of that genotype or haplotype group. The patient could then be classified or assigned to a particular genotype or haplotype group based on how similar the measured levels were compared to the control levels for a given group.

Computer System for Storing or Displaying Polymorphism Data. The invention also provides a computer system for storing and displaying polymorphism data determined for the gene. The computer system comprises a computer processing unit, a display, and a database containing the polymorphism data. The polymorphism data includes the polymorphisms, the genotypes and the haplotypes identified for a given gene in a reference population. In a preferred embodiment, the computer system is capable of producing a display showing haplotypes organized according to their evolutionary relationships. A computer may implement any or all analytical and mathematical operations involved in practicing the methods of the present invention. In addition, the computer may execute a program that generates views (or screens) displayed on a display device and with which the user can interact to view and analyze large amounts of information relating to the gene and its genomic variation, including chromosome location, gene structure, and gene family, gene expression data, polymorphism data, genetic sequence data, and clinical population data (e.g., data on ethnogeographic origin, clinical responses, genotypes, and haplotypes for one or more populations). The polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

Nucleic Acid-based Diagnostics. In another aspect, the invention provides SNP probes, which are useful in classifying subjects according to their types of genetic variation. The SNP probes according to the invention are oligonucleotides, which discriminate between SNPs in conventional allelic discrimination assays. In certain preferred embodiments, the oligonucleotides according to this aspect of the invention are complementary to one allele of the SNP and its flanking nucleic acid sequence(s), but not to any other allele of the SNP and its flanking nucleic acid sequence(s). Oligonucleotides according to this embodiment of the invention can discriminate between SNPs in various ways. For example, under stringent hybridization conditions, an oligonucleotide of appropriate length will hybridize to one SNP, but not to any other. The oligonucleotide may be labelled using a radiolabel or a fluorescent molecular tag. Alternatively, an oligonucleotide of appropriate length can be used as a primer for PCR, wherein the 3′ terminal nucleotide is complementary to one allele containing a SNP, but not to any other allele. In this embodiment, the presence or absence of amplification by PCR determines the haplotype of the SNP.

Genomic and cDNA fragments of the invention comprise at least one polymorphic site identified herein, have a length of at least 10 nucleotides, and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

Kits of the Invention. The invention provides nucleic acid and polypeptide detection kits useful for haplotyping and/or genotyping the gene in an individual. Such kits are useful for classifying individuals for the purpose of classifying individuals. Specifically, the invention encompasses kits for detecting the presence of a polypeptide or nucleic acid corresponding to a marker of the invention in a biological sample, e.g., any bodily fluid including, but not limited to, serum, plasma, lymph, cystic fluid, urine, stool, cerebrospinal fluid, ascites fluid or blood, and including biopsy samples of body tissue. For example, the kit can comprise a labelled compound or agent capable of detecting a polypeptide or an mRNA encoding a polypeptide corresponding to a marker of the invention in a biological sample and means for determining the amount of the polypeptide or mRNA in the sample, e.g., an antibody which binds the polypeptide or an oligonucleotide probe which binds to DNA or mRNA encoding the polypeptide. Kits can also include instructions for interpreting the results obtained using the kit.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as in the case of PCR. In a preferred embodiment, such kit may further comprise a DNA sample collecting means.

For antibody-based kits, the kit can comprise, e.g., (1) a first antibody, e.g., attached to a solid support, which binds to a polypeptide corresponding to a marker or the invention; and, optionally (2) a second, different antibody which binds to either the polypeptide or the first antibody and is conjugated to a detectable label.

For oligonucleotide-based kits, the kit can comprise, e.g., (1) an oligonucleotide, e.g., a detectably-labelled oligonucleotide, which hybridizes to a nucleic acid sequence encoding a polypeptide corresponding to a marker of the invention; or (2) a pair of primers useful for amplifying a nucleic acid molecule corresponding to a marker of the invention.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit.

Nucleic Acid Sequences of the Invention. In one aspect, the invention comprises one or more isolated polynucleotides. The invention also encompasses allelic variants of the same, that is, naturally occurring alternative forms of the isolated polynucleotides that encode mutant polypeptides that are identical, homologous or related to those encoded by the polynucleotides. Alternatively, non-naturally occurring variants may be produced by mutagenesis techniques or by direct synthesis techniques well-known in the art.

Accordingly, nucleic acid sequences capable of hybridizing at low stringency with any nucleic acid sequences encoding mutant polypeptide of the present invention are considered to be within the scope of the invention. Standard stringency conditions are well characterized in standard molecular biology cloning texts. See, for example Molecular Cloning: A Laboratory Manual, 2nd Ed., ed., Sambrook, Fritsch, & Maniatis (Cold Spring Harbor Laboratory Press, 1989); DNA Cloning, Volumes I and II, D. N. Glover, ed. (1985); Oligonucleotide Synthesis, M. J. Gait, ed. (1984); Nucleic Acid Hybridization, B. D. Hames & S. J. Higgins, eds (1984).

Recombinant Expression Vectors. Another aspect of the invention comprises vectors containing one or more nucleic acid sequences encoding a mutant polypeptide. In practicing the present invention, many conventional techniques in molecular biology, microbiology and recombinant DNA are used. These techniques are well-known and are explained in, e.g., Current Protocols in Molecular Biology, Vols. I-III, Ausubel, ed. (1997); Sambrook et al., Molecular Cloning: A Laboratory Manual, 2^{nd} Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, ed. (1986); Immobilized Cells and Enzymes (IRL Press, 1986); Perbal, A Practical Guide to Molecular Cloning; the series Methods in Enzymology, (Academic Press, Inc., 1984); Gene Transfer Vectors for Mammalian Cells, Miller & Calos, Eds. (Cold Spring Harbor Laboratory, New York, 1987); and Methods in Enzymology, Vols. 154 and 155, Wu & Grossman, and Wu, Eds., respectively.

For recombinant expression of one or more the polypeptides of the invention, the nucleic acid containing all or a portion of the nucleotide sequence encoding the polypeptide is inserted into an appropriate cloning vector, or an expression vector (i.e., a vector that contains the necessary elements for the transcription and translation of the inserted polypeptide coding sequence) by recombinant DNA techniques well-known in the art.

In the specification, “plasmid” and “vector” can be used interchangeably as the plasmid is the most commonly used form of vector. However, the invention is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound. Becker et al., Meth. Cell Biol. 43: 161-89 (1994). Within a recombinant expression vector, “operably linked” is intended to mean that the nucleotide sequence of interest is linked to the regulatory sequences in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods In Enzymology (Academic Press, San Diego, Calif., 1990). Regulatory sequences include those that direct constitutive expression of a nucleotide sequence in many types of host cell and those that direct expression of the nucleotide sequence only in certain host cells (e.g., tissue specific regulatory sequences). It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides, encoded by nucleic acids as described herein (e.g., mutant polypeptides and mutant-derived fusion polypeptides, etc.).

Polypeptide-Expressing Host Cells. Another aspect of the invention pertains to polypeptide-expressing host cells, which contain a nucleic acid encoding one or more mutant polypeptides of the invention. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. Sambrook et al. Molecular Cloning: A Laboratory Manual, 2nd ed. (Cold Spring Harbor Laboratory Press, Cold Spring Harbor, N.Y., 1989).

Vector DNA can be introduced into prokaryotic or eukaryotic cells via conventional transformation or transfection techniques. As used herein, the terms “transformation” and “transfection” are intended to refer to a variety of art recognized techniques for introducing foreign nucleic acid (e.g., DNA) into a host cell, including calcium phosphate or calcium chloride co precipitation, DEAE dextran mediated transfection, lipofection, or electroporation. Suitable methods for transforming or transfecting host cells can be found in Sambrook et al. Molecular Cloning: A Laboratory Manual, 2nd ed. (Cold Spring Harbor Laboratory Press, Cold Spring Harbor, N.Y., 1989), and other laboratory manuals. Methods such as electroporation, particle bombardment, calcium phosphate co-precipitation and viral transduction for introducing DNA into cells are known in the art; therefore, the choice of method may lie with the competence and preference of the skilled practitioner.

To prepare a recombinant cell of the invention, the desired isogene may be introduced into a host cell in a vector such that the isogene remains extrachromosomal. In such a situation, the gene will be expressed by the cell from the extrachromosomal location. In a preferred embodiment, the isogene is introduced into a cell in such a way that it recombines with the endogenous gene present in the cell. Vectors for the introduction of genes both for recombination and for extrachromosomal maintenance are known in the art, and any suitable vector or vector construct may be used in the invention.

The recombinant expression vectors of the invention can be designed for expression of mutant polypeptides in prokaryotic or eukaryotic cells. For example, mutant polypeptide can be expressed in bacterial cells such as Escherichia coli (E. coli), insect cells (using baculovirus expression vectors), fungal cells, e.g., yeast, or mammalian cells. Suitable host cells are discussed further in Goeddel, Gene Expression Technology: Methods In Enzymology (Academic Press, San Diego, Calif., 1990).

Expression of polypeptides in prokaryotes is most often carried out in E. coli with vectors containing constitutive or inducible promoters directing the expression of either fusion or non fusion polypeptides. Typical fusion expression vectors include pGEX (Pharmacia Biotech Inc; Smith & Johnson, Gene 67: 31 40 (1988)), pMAL (New England Biolabs, Beverly, Mass., USA) and pRIT5 (Pharmacia, Piscataway, N.J., USA) that fuse glutathione S transferase (GST), maltose E binding polypeptide, or polypeptide A, respectively, to the target recombinant polypeptide. Examples of suitable inducible non fusion E. coli expression vectors include pTrc (Amrann et al., Gene 69: 301 315 (1988)) and pET 11d (Studier et al., Gene Expression Technology: Methods In Enzymology (Academic Press, San Diego, Calif., 1990) pp. 60-89. Other strategies are described by Gottesman, Gene Expression Technology: Methods In Enzymology (Academic Press, San Diego, Calif., 1990) pp. 119-128 and by Wada, et al., Nucl. Acids Res. 20: 2111 2118 (1992)).

The polypeptide expression vector may be a yeast expression vector. Examples of vectors for expression in yeast Saccharomyces cerivisae include pYepSec1 (Baldari et al., EMBO J. 6: 229 234 (1987)), pMFa (Kurjan & Herskowitz, Cell 30: 933-943 (1982)), pJRY 88 (Schultz et al., Gene 54: 113 123 (1987)), pYES2 (InVitrogen Corporation, San Diego, Calif. USA), and picZ (InVitrogen Corp, San Diego, Calif., USA). Alternatively, mutant polypeptide can be expressed in insect cells using baculovirus expression vectors. Baculovirus vectors available for expression of polypeptides in cultured insect cells (e.g., SF9 cells) include the pAc series (Smith et al., Mol. Cell. Biol. 3: 2156 2165 (1983)) and the pVL series (Lucklow & Summers, Virology 170: 31 39 (1989)). The nucleic acid of the invention may be expressed in mammalian cells using a mammalian expression vector such as pCDM8 (Seed, Nature 329: 842 846 (1987)) or pMT2PC (Kaufman et al., EMBO J. 6: 187 195 (1987)). For other suitable expression systems for both prokaryotic and eukaryotic cells see, e.g., Chapters 16 and 17 of Sambrook et al., Molecular Cloning: A Laboratory Manual, 2nd ed. (Cold Spring Harbor Laboratory Press, Cold Spring Harbor, N.Y., 1989). Tissue specific and developmentally-regulated regulatory elements are known in the art. For a discussion of the regulation of gene expression using antisense genes see, e.g., Weintraub et al., “Antisense RNA as a molecular tool for genetic analysis,” Trends in Genetics 1(1) (1986).

A host cell that includes a compound of the invention, such as a prokaryotic or eukaryotic host cell in culture, can be used to produce (i.e., express) recombinant mutant polypeptide. Purification of recombinant polypeptides is well-known in the art and includes ion exchange purification techniques, or affinity purification techniques, for example with an antibody to the compound.

Transgenic Animals. Recombinant organisms, i.e., transgenic animals, expressing a variant gene of the invention are prepared using standard procedures known in the art. Transgenic animals carrying the constructs of the invention can be made by several methods known to those having skill in the art. See, e.g., U.S. Pat. No. 5,610,053 and “The Introduction of Foreign Genes into Mice” and the cited references therein, in: Recombinant DNA, Eds. J. D. Watson, M. Gilman, J. Witkowski & M. Zoller (W.H. Freeman and Company, New York) pp. 254-272. Transgenic animals stably expressing a human isogene and producing human protein can be used as biological models for studying diseases related to abnormal expression and/or activity, and for screening and assaying various candidate drugs, compounds, and treatment regimens to reduce the symptoms or effects of these diseases.

Characterizing Gene Expression Level. Methods to detect and measure mRNA levels (i.e., gene transcription level) and levels of polypeptide gene expression products (i.e., gene translation level) are well-known in the art and include the use of nucleotide microarrays and polypeptide detection methods involving mass spectrometers and/or antibody detection and quantification techniques. See also, Tom Strachan & Andrew Read, Human Molecular Genetics, 2^{nd} Edition. (John Wiley and Sons, Inc. Publication, New York, 1999)).

Determination of Target Gene Transcription. The determination of the level of the expression product of the gene in a biological sample, e.g., the tissue or body fluids of an individual, may be performed in a variety of ways. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells. See, e.g., Ausubel et al., Ed., Curr. Prot. Mol. Biol. (John Wiley & Sons, New York, 1987-1999).

In one embodiment, the level of the mRNA expression product of the target gene is determined. Methods to measure the level of a specific mRNA are well-known in the art and include Northern blot analysis, reverse transcription PCR and real time quantitative PCR or by hybridization to a oligonucleotide array or microarray. In other more preferred embodiments, the determination of the level of expression may be performed by determination of the level of the protein or polypeptide expression product of the gene in body fluids or tissue samples including but not limited to blood or serum. Large numbers of tissue samples can readily be processed using techniques well-known to those of skill in the art, such as, e.g., the single-step RNA isolation process of U.S. Pat. No. 4,843,155.

The isolated mRNA can be used in hybridization or amplification assays that include, but are not limited to, Southern or Northern analyses, PCR analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a marker of the present invention. Other suitable probes for use in the diagnostic assays of the invention are described herein. Hybridization of an mRNA with the probe indicates that the marker in question is being expressed.

In one format, the probes are immobilized on a solid surface and the mRNA is contacted with the probes, for example, in an Affymetrix gene chip array (Affymetrix, Calif. USA). A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by RT-PCR (the experimental embodiment set forth in U.S. Pat. No. 4,683,202); ligase chain reaction (Barany et al., Proc. Natl. Acad. Sci. USA 88:189-193 (1991)) self-sustained sequence replication (Guatelli et al., Proc. Natl. Acad. Sci. USA 87: 1874-1878 (1990)); transcriptional amplification system (Kwoh et al., Proc. Natl. Acad. Sci. USA 86: 1173-1177 (1989)); Q-Beta Replicase (Lizardi et al., Biol. Technology 6: 1197 (1988)); rolling circle replication (U.S. Pat. No. 5,854,033); or any other nucleic acid amplification method, followed by the detection of the amplified molecules using techniques well-known to those of skill in the art. These detection schemes are especially useful for the detection of the nucleic acid molecules if such molecules are present in very low numbers. As used herein, “amplification primers” are defined as being a pair of nucleic acid molecules that can anneal to 5′ or 3′ regions of a gene (plus and minus strands, respectively, or vice-versa) and contain a short region in between. In general, amplification primers are from about 10-30 nucleotides in length and flank a region from about 50-200 nucleotides in length.

Real-time quantitative PCR(RT-PCR) is one way to assess gene expression levels, e.g., of genes of the invention, e.g., those containing SNPs and polymorphisms of interest. The RT-PCR assay utilizes an RNA reverse transcriptase to catalyze the synthesis of a DNA strand from an RNA strand, including an mRNA strand. The resultant DNA may be specifically detected and quantified and this process may be used to determine the levels of specific species of mRNA. One method for doing this is TAQMAN® (PE Applied Biosystems, Foster City, Calif., USA) and exploits the 5′ nuclease activity of AMPLITAQ GOLD™ DNA polymerase to cleave a specific form of probe during a PCR reaction. This is referred to as a TAQMAN™ probe. See Luthra et al., Am. J. Pathol. 153: 63-68 (1998); Kuimelis et al., Nucl. Acids Symp. Ser. 37: 255-256 (1997); and Mullah et al., Nucl. Acids Res. 26(4): 1026-1031 (1998)). During the reaction, cleavage of the probe separates a reporter dye and a quencher dye, resulting in increased fluorescence of the reporter. The accumulation of PCR products is detected directly by monitoring the increase in fluorescence of the reporter dye. Heid et al., Genome Res. 6(6): 986-994 (1996)). The higher the starting copy number of nucleic acid target, the sooner a significant increase in fluorescence is observed. See Gibson, Heid & Williams et al., Genome Res. 6: 995-1001 (1996).

Other technologies for measuring the transcriptional state of a cell produce pools of restriction fragments of limited complexity for electrophoretic analysis, such as methods combining double restriction enzyme digestion with phasing primers (see, e.g., EP 0 534858 A1), or methods selecting restriction fragments with sites closest to a defined mRNA end. (See, e.g., Prashar & Weissman, Proc. Natl. Acad. Sci. USA 93(2) 659-663 (1996)).

Other methods statistically sample cDNA pools, such as by sequencing sufficient bases, e.g., 20-50 bases, in each of multiple cDNAs to identify each cDNA, or by sequencing short tags, e.g., 9-10 bases, which are generated at known positions relative to a defined mRNA end pathway pattern. See, e.g., Velculescu, Science 270: 484-487 (1995). The cDNA levels in the samples are quantified and the mean, average and standard deviation of each cDNA is determined using by standard statistical means well-known to those of skill in the art. Norman T. J. Bailey, Statistical Methods In Biology, 3rd Edition (Cambridge University Press, 1995).

Detection of Polypeptides. Immunological Detection Methods. Expression of the protein encoded by the genes of the invention can be detected by a probe which is detectably labelled, or which can be subsequently labelled. The term “labelled”, with regard to the probe or antibody, is intended to encompass direct-labelling of the probe or antibody by coupling, i.e., physically linking, a detectable substance to the probe or antibody, as well as indirect-labelling of the probe or antibody by reactivity with another reagent that is directly-labelled. Examples of indirect labelling include detection of a primary antibody using a fluorescently-labelled secondary antibody and end-labelling of a DNA probe with biotin such that it can be detected with fluorescently-labelled streptavidin. Generally, the probe is an antibody that recognizes the expressed protein. A variety of formats can be employed to determine whether a sample contains a target protein that binds to a given antibody. Immunoassay methods useful in the detection of target polypeptides of the present invention include, but are not limited to, e.g., dot blotting, western blotting, protein chips, competitive and non-competitive protein binding assays, enzyme-linked immunosorbant assays (ELISA), immunohistochemistry, fluorescence activated cell sorting (FACS), and others commonly used and widely-described in scientific and patent literature, and many employed commercially. A skilled artisan can readily adapt known protein/antibody detection methods for use in determining whether cells express a marker of the present invention and the relative concentration of that specific polypeptide expression product in blood or other body tissues. Proteins from individuals can be isolated using techniques that are well-known to those of skill in the art. The protein isolation methods employed can, e.g., be such as those described in Harlow & Lane, Antibodies: A Laboratory Manual (Cold Spring Harbor Laboratory Press, Cold Spring Harbor, N.Y., 1988)).

For the production of antibodies to a protein encoded by one of the disclosed genes, various host animals may be immunized by injection with the polypeptide, or a portion thereof. Such host animals may include, but are not limited to, rabbits, mice and rats. Various adjuvants may be used to increase the immunological response, depending on the host species including, but not limited to, Freund's (complete and incomplete), mineral gels, such as aluminium hydroxide; surface active substances, such as lysolecithin, pluronic polyols, polyanions, peptides, oil emulsions, keyhole limpet haemocyanin and dinitrophenol; and potentially useful human adjuvants, such as bacille Camette-Guerin (BCG) and Corynebacterium parvum.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler & Milstein, Nature 256: 495-497 (1975); and U.S. Pat. No. 4,376,110; the human B-cell hybridoma technique of Kosbor et al., Immunol. Today 4: 72 (1983); Cole et al., Proc. Natl. Acad. Sci. USA 80: 2026-2030 (1983); and the EBV-hybridoma technique of Cole et al., Monoclonal Antibodies and Cancer Therapy (Alan R. Liss, Inc., 1985) pp. 77-96.

In addition, techniques developed for the production of “chimeric antibodies” (see Morrison et al., Proc. Natl. Acad. Sci. USA 81: 6851-6855 (1984); Neuberger et al., Nature 312: 604-608 (1984); and Takeda et al., Nature 314: 452-454 (1985)), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies (U.S. Pat. No. 4,946,778; Bird, Science 242: 423-426 (1988); Huston et al., Proc. Natl. Acad. Sci. USA 85: 5879-5883 (1988); and Ward et al., Nature 334: 544-546 (1989)) can be adapted to produce differentially expressed gene single-chain antibodies.

Techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibodies or antibody fragments can be used in methods, such as Western blots or immunofluorescence techniques, to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

A useful method, for ease of detection, is the sandwich ELISA, of which a number of variations exist, all of which are intended to be used in the methods and assays of the present invention. As used herein, “sandwich assay” is intended to encompass all variations on the basic two-site technique. Immunofluorescence and EIA techniques are both very well-established in the art. However, other reporter molecules, such as radioisotopes, chemiluminescent or bioluminescent molecules may also be employed. It will be readily apparent to the skilled artisan how to vary the procedure to suit the required use.

Whole genome monitoring of protein, i.e., the “proteome,” can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. As noted above, methods for making monoclonal antibodies are well-known. See, e.g., Harlow & Lane, Antibodies: A Laboratory Manual” (Cold Spring Harbor Laboratory Press, Cold Spring Harbor, N.Y., 1988)). In a preferred embodiment, monoclonal antibodies are raised against synthetic peptide fragments designed based on genomic sequence of the cell. With such an antibody array, proteins from the cell are contacted to the array and their binding is measured with assays known in the art.

Detection of Polypeptides. Two-Dimensional Gel Electrophoresis. Two-dimensional gel electrophoresis is well-known in the art and typically involves isoelectric focusing along a first dimension followed by SDS-PAGE electrophoresis along a second dimension. See, e.g., Hames et al., Gel Electrophoresis of Proteins: A Practical Approach (IRL Press, New York, 1990); Shevchenko et al., Proc. Natl. Acad. Sci. USA 93: 14440-14445 (1996); Sagliocco et al., Yeast 12: 1519-1533 (1996); and Lander, Science 274: 536-539 (1996).

Detection of Polypeptides. Mass Spectroscopy. The identity as well as expression level of target polypeptide can be determined using mass spectroscopy technique (MS). MS-based analysis methodology is useful for analysis of isolated target polypeptide as well as analysis of target polypeptide in a biological sample. MS formats for use in analyzing a target polypeptide include ionization (I) techniques, such as, but not limited to, matrix assisted laser desorption (MALDI), continuous or pulsed electrospray ionization (ESI) and related methods, such as ionspray or thermospray, and massive cluster impact (MCI). Such ion sources can be matched with detection formats, including linear or non-linear reflectron time of flight (TOF), single or multiple quadrupole, single or multiple magnetic sector, Fourier transform ion cyclotron resonance (FTICR), ion trap and combinations thereof such as ion-trap/TOF. For ionization, numerous matrix/wavelength combinations (e.g., matrix assisted laser desorption (MALDI)) or solvent combinations (e.g., ESI) can be employed.

For mass spectroscopy (MS) analysis, the target polypeptide can be solubilised in an appropriate solution or reagent system. The selection of a solution or reagent system, e.g., an organic or inorganic solvent, will depend on the properties of the target polypeptide and the type of MS performed, and is based on methods well-known in the art. See, e.g., Vorm et al., Anal. Chem. 61: 3281 (1994) for MALDI; and Valaskovic et al., Anal. Chem. 67: 3802 (1995), for ESI. MS of peptides also is described, e.g., in International PCT Application No. WO 93/24834 and U.S. Pat. No. 5,792,664. A solvent is selected that minimizes the risk that the target polypeptide will be decomposed by the energy introduced for the vaporization process. A reduced risk of target polypeptide decomposition can be achieved, e.g., by embedding the sample in a matrix. A suitable matrix can be an organic compound such as a sugar, e.g., a pentose or hexose, or a polysaccharide such as cellulose. Such compounds are decomposed thermolytically into CO₂ and H₂O such that no residues are formed that can lead to chemical reactions. The matrix also can be an inorganic compound, such as nitrate of ammonium, which is decomposed essentially without leaving any residue. Use of these and other solvents is known to those of skill in the art. See, e.g., U.S. Pat. No. 5,062,935. Electrospray MS has been described by Fenn et al., J. Phys. Chem. 88: 4451-4459 (1984); and PCT Application No. WO 90/14148; and current applications are summarized in review articles. See Smith et al., Anal. Chem. 62: 882-89 (1990); and Ardrey, Spectroscopy 4: 10-18 (1992).

The mass of a target polypeptide determined by MS can be compared to the mass of a corresponding known polypeptide. For example, where the target polypeptide is a mutant protein, the corresponding known polypeptide can be the corresponding non-mutant protein, e.g., wild-type protein. With ESI, the determination of molecular weights in femtomole amounts of sample is very accurate due to the presence of multiple ion peaks, all of which can be used for mass calculation. Sub-attomole levels of protein have been detected, e.g., using ESI MS (Valaskovic et al., Science 273: 1199-1202 (1996)) and MALDI MS (Li et al., J. Am. Chem. Soc. 118: 1662-1663 (1996)).

Matrix Assisted Laser Desorption (MALDI). The level of the target protein in a biological sample, e.g., body fluid or tissue sample, may be measured by means of mass spectrometric (MS) methods including, but not limited to, those techniques known in the art as matrix-assisted laser desorption/ionization, time-of-flight mass spectrometry (MALDI-TOF-MS) and surfaces enhanced for laser desorption/ionization, time-of-flight mass spectrometry (SELDI-TOF-MS) as further detailed below. Methods for performing MALDI are well-known to those of skill in the art. See, e.g., Juhasz et al., Analysis, Anal. Chem. 68: 941-946 (1996), and see also, e.g., U.S. Pat. Nos. 5,777,325; 5,742,049; 5,654,545; 5,641,959; 5,654,545 and 5,760,393 for descriptions of MALDI and delayed extraction protocols. Numerous methods for improving resolution are also known. MALDI-TOF-MS has been described by Hillenkamp et al., Biological Mass Spectrometry, Burlingame & McCloskey, eds. (Elsevier Science Publ., Amsterdam, 1990) pp. 49-60.

A variety of techniques for marker detection using mass spectroscopy can be used. See Bordeaux Mass Spectrometry Conference Report, Hillenkamp, Ed., pp. 354-362 (1988); Bordeaux Mass Spectrometry Conference Report, Karas & Hillenkamp, Eds., pp. 416-417 (1988); Karas & Hillenkamp, Anal. Chem. 60: 2299-2301 (1988); and Karas et al., Biomed. Environ. Mass Spectrum 18: 841-843 (1989). The use of laser beams in TOF-MS is shown, e.g., in U.S. Pat. Nos. 4,694,167; 4,686,366, 4,295,046 and 5,045,694, which are incorporated herein by reference in their entireties. Other MS techniques allow the successful volatilization of high molecular weight biopolymers, without fragmentation, and have enabled a wide variety of biological macromolecules to be analyzed by mass spectrometry.

Surfaces Enhanced for Laser Desorption/Ionization (SELDI). Other techniques are used which employ new MS probe element compositions with surfaces that allow the probe element to actively participate in the capture and docking of specific analytes, described as Affinity Mass Spectrometry (AMS). See SELDI patents U.S. Pat. Nos. 5,719,060; 5,894,063; 6,020,208; 6,027,942; 6,124,137; and U.S. Patent application No. U.S. 2003/0003465. Several types of new MS probe elements have been designed with Surfaces Enhanced for Affinity Capture (SEAC). See Hutchens & Yip, Rapid Commun. Mass Spectrom. 7: 576-580 (1993). SEAC probe elements have been used successfully to retrieve and tether different classes of biopolymers, particularly proteins, by exploiting what is known about protein surface structures and biospecific molecular recognition. The immobilized affinity capture devices on the MS probe element surface, i.e., SEAC, determines the location and affinity (specificity) of the analyte for the probe surface, therefore the subsequent analytical MS process is efficient.

Within the general category of SELDI are three separate subcategories: (1) Surfaces Enhanced for Neat Desorption (SEND), where the probe element surfaces, i.e., sample presenting means, are designed to contain Energy Absorbing Molecules (EAM) instead of “matrix” to facilitate desorption/ionizations of analytes added directly (neat) to the surface. (2) SEAC, where the probe element surfaces, i.e., sample presenting means, are designed to contain chemically defined and/or biologically defined affinity capture devices to facilitate either the specific or non-specific attachment or adsorption (so-called docking or tethering) of analytes to the probe surface, by a variety of mechanisms (mostly non-covalent). (3) Surfaces Enhanced for Photolabile Attachment and Release (SEPAR), where the probe element surfaces, i.e., sample presenting means, are designed or modified to contain one or more types of chemically defined cross-linking molecules to serve as covalent docking devices. The chemical specificities determining the type and number of the photolabile molecule attachment points between the SEPAR sample presenting means (i.e., probe element surface) and the analyte (e.g., protein) may involve any one or more of a number of different residues or chemical structures in the analyte (e.g., His, Lys, Arg, Tyr, Phe and Cys residues in the case of proteins and peptides).

Functionalizing Polypeptides. A polypeptide of interest also can be modified to facilitate conjugation to a solid support. A chemical or physical moiety can be incorporate into the polypeptide at an appropriate, position. For example, a polypeptide of interest can be modified by adding an appropriate functional group to the carboxyl terminus or amino terminus of the polypeptide, or to an amino acid in the peptide, (e.g., to a reactive side chain, or to the peptide backbone. The artisan will recognize, however, that such a modification, e.g., the incorporation of a biotin moiety, can affect the ability of a particular reagent to interact specifically with the polypeptide and, accordingly, will consider this factor, if relevant, in selecting how best to modify a polypeptide of interest. A naturally-occurring amino acid normally present in the polypeptide also can contain a functional group suitable for conjugating the polypeptide to the solid support. For example, a cysteine residue present in the polypeptide can be used to conjugate the polypeptide to a support containing a sulfhydryl group through a disulfide linkage, e.g., a support having cysteine residues attached thereto. Other bonds that can be formed between two amino acids, include, but are not limited to, e.g., monosulfide bonds between two lanthionine residues, which are non-naturally-occurring amino acids that can be incorporated into a polypeptide; a lactam bond formed by a transamidation reaction between the side chains of an acidic amino acid and a basic amino acid, such as between the y-carboxyl group of Glu (or alpha carboxyl group of Asp) and the amino group of Lys; or a lactone bond produced, e.g., by a crosslink between the hydroxy group of Ser and the carboxyl group of Glu (or alpha carboxyl group of Asp). Thus, a solid support can be modified to contain a desired amino acid residue, e.g., a Glu residue, and a polypeptide having a Ser residue, particularly a Ser residue at the N-terminus or C-terminus, can be conjugated to the solid support through the formation of a lactone bond. The support need not be modified to contain the particular amino acid, e.g., Glu, where it is desired to form a lactone-like bond with a Ser in the polypeptide, but can be modified, instead, to contain an accessible carboxyl group, thus providing a function corresponding to the alpha carboxyl group of Glu.

Thiol-Reactive Functionalities. A thiol-reactive functionality is particularly useful for conjugating a polypeptide to a solid support. A thiol-reactive functionality is a chemical group that can rapidly react with a nucleophilic thiol moiety to produce a covalent bond, e.g., a disulfide bond or a thioether bond. A variety of thiol-reactive functionalities are known in the art, including, e.g., haloacetyls, such as iodoacetyl; diazoketones; epoxy ketones, alpha- and beta-unsaturated carbonyls, such as alpha-enones and beta-enones; and other reactive Michael acceptors, such as maleimide; acid halides; benzyl halides; and the like. See Greene & Wuts, Protective Groups in Organic Synthesis, 2^{nd} Edition (John Wiley & Sons, 1991).

If desired, the thiol groups can be blocked with a photocleavable protecting group, which then can be selectively cleaved, e.g., by photolithography, to provide portions of a surface activated for immobilization of a polypeptide of interest. Photocleavable protecting groups are known in the art (see, e.g., published International PCT Application No. WO 92/10092; and McCray et al., Ann. Rev. Biophys. Biophys. Chem. 18: 239-270 (1989)) and can be selectively de-blocked by irradiation of selected areas of the surface using, e.g., a photolithography mask.

Linkers. A polypeptide of interest can be attached directly to a support via a linker. Any linkers known to those of skill in the art to be suitable for linking peptides or amino acids to supports, either directly or via a spacer, may be used. For example, the polypeptide can be conjugated to a support, such as a bead, through means of a variable spacer. Linkers, include, Rink amide linkers (see, e.g., Rink, Tetrahedron Lett. 28: 3787 (1976)); trityl chloride linkers (see, e.g., Leznoff, Ace Chem. Res. 11: 327 (1978)); and Merrifield linkers (see, e.g., Bodansky et al., Peptide Synthesis, 2^{nd} Edition (Academic Press, New York, 1976)). For example, trityl linkers are known. See, e.g., U.S. Pat. Nos. 5,410,068 and 5,612,474. Amino trityl linkers are also known. See, e.g., U.S. Pat. No. 5,198,531. Other linkers include those that can be incorporated into fusion proteins and expressed in a host cell. Such linkers may be selected amino acids, enzyme substrates or any suitable peptide. The linker may be made, e.g., by appropriate selection of primers when isolating the nucleic acid. Alternatively, they may be added by post-translational modification of the protein of interest. Linkers that are suitable for chemically linking peptides to supports, include disulfide bonds, thioether bonds, hindered disulfide bonds and covalent bonds between free reactive groups, such as amine and thiol groups.

Cleavable Linkers. A linker can provide a reversible linkage such that it is cleaved under the select conditions. In particular, selectively cleavable linkers, including photocleavable linkers (see U.S. Pat. No. 5,643,722), acid cleavable linkers (see Fattom et al., Infect. Immun. 60: 584-589 (1992)), acid-labile linkers (see Welhöner et al., J. Biol. Chem. 266: 4309-4314 (1991)) and heat sensitive linkers are useful. A linkage can be, e.g., a disulfide bond, which is chemically cleavable by mercaptoethanol or dithioerythrol; a biotin/streptavidin linkage, which can be photocleavable; a heterobifunctional derivative of a trityl ether group, which can be cleaved by exposure to acidic conditions or under conditions of MS (see Köster et al, Tetrahedron Lett. 31: 7095 (1990)); a levulinyl-mediated linkage, which can be cleaved under almost neutral conditions with a hydrazinium/acetate buffer; an arginine-arginine or a lysine-lysine bond, either of which can be cleaved by an endopeptidase, such as trypsin; a pyrophosphate bond, which can be cleaved by a pyrophosphatase; or a ribonucleotide bond, which can be cleaved using a ribonuclease or by exposure to alkali condition. A photolabile cross-linker, such as 3-amino-(2-nitrophenyl)propionic acid can be employed as a means for cleaving a polypeptide from a solid support. Brown et al., Mol Divers, pp. 4-12 (1995); Rothschild et al., Nucl. Acids. Res. 24: 351-66 (1996); and U.S. Pat. No. 5,643,722. Other linkers include RNA linkers that are cleavable by ribozymes and other RNA enzymes and linkers, such as the various domains, such as CH₁, CH₂ and CH₃, from the constant region of human IgG1. See, Batra et al., Mol Immunol 30: 379-396 (1993).

Combinations of any linkers are also contemplated herein. For example, a linker that is cleavable under MS conditions, such as a silyl linkage or photocleavable linkage, can be combined with a linker, such as an avidin biotin linkage, that is not cleaved under these conditions, but may be cleaved under other conditions. Acid-labile linkers are particularly useful chemically cleavable linkers for mass spectrometry, especially for MALDI-TOF, because the acid labile bond is cleaved during conditioning of the target polypeptide upon addition of a 3-HPA matrix solution. The acid labile bond can be introduced as a separate linker group, e.g., an acid labile trityl group, or can be incorporated in a synthetic linker by introducing one or more silyl bridges using diisopropylysilyl, thereby forming a diisopropylysilyl linkage between the polypeptide and the solid support. The diisopropylysilyl linkage can be cleaved using mildly acidic conditions, such as 1.5% trifluoroacetic acid (TFA) or 3-HPA/1% TFA MALDI-TOF matrix solution. Methods for the preparation of diisopropylysilyl linkages and analogues thereof are well-known in the art. See, e.g., Saha et al., J. Org. Chem. 58: 7827-7831 (1993).

Use of a Pin Tool to Immobilize a Polypeptide. The immobilization of a polypeptide of interest to a solid support using a pin tool can be particularly advantageous. Pin tools include those disclosed herein or otherwise known in the art. See, e.g., U.S. application Ser. Nos. 08/786,988 and 08/787,639; and International PCT Application No. WO 98/20166.

A pin tool in an array, e.g., a 4×4 array, can be applied to wells containing polypeptides of interest. Where the pin tool has a functional group attached to each pin tip, or a solid support, e.g., functionalized beads or paramagnetic beads are attached to each pin, the polypeptides in a well can be captured (1 pmol capacity). During the capture step, the pins can be kept in motion (vertical, 1-2 mm travel) to increase the efficiency of the capture. Where a reaction, such as an in vitro transcription is being performed in the wells, movement of the pins can increase efficiency of the reaction. Further immobilization can result by applying an electrical field to the pin tool. When a voltage is applied to the pin tool, the polypeptides are attracted to the anode or the cathode, depending on their net charge.

For more specificity, the pin tool (with or without voltage) can be modified to have conjugated thereto a reagent specific for the polypeptide of interest, such that only the polypeptides of interest are bound by the pins. For example, the pins can have nickel ions attached, such that only polypeptides containing a polyhistidine sequence are bound. Similarly, the pins can have antibodies specific for a target polypeptide attached thereto, or to beads that, in turn, are attached to the pins, such that only the target polypeptides, which contain the epitope recognized by the antibody, are bound by the pins.

Captured polypeptides can be analyzed by a variety of means including, e.g., spectrometric techniques, such as UV/VIS, IR, fluorescence, chemiluminescence, NMR spectroscopy, MS or other methods known in the art, or combinations thereof. If conditions preclude direct analysis of captured polypeptides, the polypeptides can be released or transferred from the pins, under conditions such that the advantages of sample concentration are not lost. Accordingly, the polypeptides can be removed from the pins using a minimal volume of eluent, and without any loss of sample. Where the polypeptides are bound to the beads attached to the pins, the beads containing the polypeptides can be removed from the pins and measurements made directly from the beads.

Pin tools can be useful for immobilizing polypeptides of interest in spatially addressable manner on an array. Such spatially addressable or pre-addressable arrays are useful in a variety of processes, including, for example, quality control and amino acid sequencing diagnostics. The pin tools described in the U.S. application Ser. Nos. 08/786,988 and 08/787,639 and International PCT Application No. WO 98/20166 are serial and parallel dispensing tools that can be employed to generate multi-element arrays of polypeptides on a surface of the solid support. The array surface can be flat, with beads or geometrically altered to include wells, which can contain beads. In addition, MS geometries can be adapted for accommodating a pin tool apparatus.

Other Aspects of the Biological State. In various embodiments of the invention, aspects of the biological activity state, or mixed aspects can be measured in order to obtain drug and pathway responses. The activities of proteins relevant to the characterization of cell function can be measured, and embodiments of this invention can be based on such measurements. Activity measurements can be performed by any functional, biochemical or physical means appropriate to the particular activity being characterized. Where the activity involves a chemical transformation, the cellular protein can be contacted with natural substrates, and the rate of transformation measured. Where the activity involves association in multimeric units, e.g., association of an activated DNA binding complex with DNA, the amount of associated protein or secondary consequences of the association, such as amounts of mRNA transcribed, can be measured. Also, where only a functional activity is known, e.g., as in cell cycle control, performance of the function can be observed. However known and measured, the changes in protein activities form the response data analyzed by the methods of this invention. In alternative and non-limiting embodiments, response data may be formed of mixed aspects of the biological state of a cell. Response data can be constructed from, e.g., changes in certain mRNA abundances, changes in certain protein abundances and changes in certain protein activities.

The following EXAMPLE is presented in order to more fully illustrate the preferred embodiments of the invention. This EXAMPLE should in no way be construed as limiting the scope of the invention, as defined by the appended claims.

### Example I

### Affymetrix 100K Genome Scan in Patients from the InDDEx Trial

Introduction and summary. The purpose of this EXAMPLE is to describe genetic association studies identifying twenty-five (25) AD-associated mutations of the invention. Genome-wide analyses for genetic association studies have only become possible within the past two years. The Investigation Into Delay to Diagnosis of Alzheimer's Disease With Exelon (InDDex; ENA713B IA07) clinical trial followed patients suffering from MCI and given either Exelon (rivastigmine) at various doses or placebo and followed their conversion to AD. The trial had an optional DNA collection component, in which 442 samples were collected.

To identify genetic variation contributing to MCI to AD conversion, 420 patient samples from the InDDex (ENA713B IA07) clinical trial were genotyped using the Affymetrix 100K genotyping platform and whole-genome association analysis was performed, on single points and inferred haplotype blocks, using the case-control test of Haploview. The outcome phenotype was conversion or non-conversion from MCI to AD. The unbiased, genome-wide approach of the present studies led to the identification of mutations and genetic regions that have not been previously linked to AD.

Commonly used criteria for a diagnosis of amnestic MCI include, but are not limited to, e.g., deficient memory; essentially normal judgment, perception and reasoning skills; largely normal activities of daily living; reduced performance on memory tests compared to other people of similar age and educational background; and absence of dementia. The risk factors for amnestic (memory-related) MCI are the same as those for Alzheimer's and include family history; genetics and age. MCI is contrasted with AD, which is the loss of intellectual and social abilities severe enough to interfere with daily functioning. Dementia occurs in people with Alzheimer's disease because healthy brain tissue degenerates, causing a steady decline in memory and mental abilities.

Methods. DNA was extracted from collected blood samples. Target preparation, hybridization to microarrays, scanning and data capture were all performed as described in the Affymetrix protocols for the 100K platform. Ultimately, 420 of the 442 DNA samples were successfully genotyped, with the other 22 dropped for reasons of DNA quality or quantity.

Genotype data were uploaded from GCOS in the chip hybridization facility to the BMD server, and exported to a Linux server in Haploview .ped input format, along with matching .info format annotation files. (Samples with a call rate of <90% or more than one mismatch between the duplicated HindIII and XbaI assays were excluded from the datasets.) Outcome (conversion/non-conversion) data from the ENA713B IA07 clinical database were merged into the genotype data and case-control analysis was performed on a per-chromosome basis in Haploview with the following parameters: “-nogui-check-assocCC-blockoutput GAB-hwcutoff 0.00001-maxDistance 200”. Otherwise, default settings were used. The per-chromosome output files were assembled into single genome-wide files, imported to a Windows server and merged in SAS.

Top results, both single-point and haplotype blocks were ranked by the provided Haploview chi-square value, with single-point results also ranked by a Fisher's Exact Test, and annotated with assay data from the Affymetrix database. None of the single-point results meet study-wide significance (p=0.05) after a Bonferroni penalty for multiple tests (n=109780 informative tests), nor do any of the combined single-point and haplotype tests meet a study-wide significance threshold (p=0.05) in permutation testing in Haploview.

Results. Single marker and haplotype analysis was performed across the genome, associating allele or haplotypes with AD conversion. Results were stratified by gender, APOE E4 status, and treatment arm. The Affymetrix 100K Genotyping Chip utilized in the present studies is similar in mechanism to the microarrays used for expression profiling. The set of two chips used in these studies provided genotype data for >100,000 SNPs evenly distributed across the genome. Four hundred twenty (420) of 442 samples from the Exelon InDDex trial were successfully hybridized to the genotyping chips used in the present studies. Genome complexity was reduced via digestion with restriction enzymes and amplified, hybridized to chips and scanned. Some 4.8 million genotypes were generated.

Genotype QC steps. Affymetrix 100K Array was evaluated by comparison of 30 redundant SNPs and comparison of gender between XbaI and HindIII pairs. A chip was eliminated if there was more than one mismatch. A sample was eliminated if >1 missing chip. Chips with “per sample call rates” of ≧90% were included in the analysis. A summary of the performance of the Affymetrix 100K Array is provided in TABLE 2A below.

TABLE 2A
Affymetrix 100K Array performance

| | First Pass Experiment | Repeats |
|---|---|---|
| Number of patients: | 442 | |
| Number of DNA preps: | 884 (2 chips/patient) | 159 |
| Number of DNA prep success: | 793 (89.70%) | 136 (85.5%) |
| Number of DNA put on chips: | 793 | 124 |
| Number of passed chips: | 758 (95.5%) | 122 (98%) |
| Average call rate per chip: | 96% | 96.6% |

The number of patient samples with 2 successful chips was 420 (95%). The number of patient samples with 1 failed assay/chip was 17 (3.9%). The number of patient samples with 2 failed assays/chips was 5 (1.1%). The observed reproducibility of assay performance for the studies of the invention was 99.9%. Eight samples of an Affymetrix-provided positive control also indicate a 99.9% accuracy rate for called genotypes.

As shown in TABLE 2B, there were no significant differences between the entire clinical study and the genome scan study for demographic characteristics, baseline MMSE, and AD conversion rate.

TABLE 2B
Demographic Characteristics and Primary Clinical Endpoint

| | All patients* | | Genome scan population** | |
|---|---|---|---|---|
| Variable | Exelon | Placebo | Exelon | Placebo |
| Number of | n = 508 | n = 510 | n = 211 | n = 208 |
| subjects | | | | |
| Age | 70.30 (7.35) | 70.63 (7.60) | 70.82 (7.29) | 70.61 (7.54) |
| Gender, | 53.15 | 51.37 | 53.08 | 48.08 |
| Female % | | | | |
| Race, | 97.24 | 96.86 | 94.79 | 95.19 |
| Caucasians % | | | | |
| Education, | 10.97 (4.01) | 11.09 (4.15) | 11.59 (4.10) | 11.65 (4.50) |
| years | | | | |
| MMSE at | 27.02 (2.64) | 26.90 (2.79) | 26.82 (2.78) | 26.85 (2.81) |
| baseline | | | | |
| Converted | 17.32 | 21.37 | 17.06 | 17.31 |
| to AD, % | | | | |

Values are mean (SD).
*Patients enrolled in and completed the clinical study.
**Patients consented for pharmacogenetic study and successfully passed the 100K scan.

Haplotype Analysis. For each haplotype, all haplotype frequencies for cases and controls were estimated. Each haplotype was tested for association with AD conversion. All associations were ranked by p-value (chi-square) to prioritize for follow-up. For the haplotype block assessment, 16,858 haplotype blocks were identified across the genome, utilizing the method of (Gabriel et al., Science 296:2225-2229 (2002))

Fifteen (15) top associations observed by haplotype block assessment (i.e., haplotypes of the invention) are summarized below in TABLE 3.

TABLE 3

| Chromo- | | | Major | | | | Fisher |
|---|---|---|---|---|---|---|---|
| some | Haplotype | Frequency | alleles | Case, Control Ratios | Chi-square | p-value | p-value |
| 7 | Block 357-AABBBBBB | 0.015 | | 8.0:132.0, 4.6:675.4 | 19.771 | 8.7308E−06 | |
| | SNP_A-1755251 | | A, A | 123:17, 617:63 | 1.092 | 0.296 | 0.278 |
| | SNP_A-1651447 | | A, A | 102:34, 491:167 | 0.009 | 0.926 | 1.000 |
| | SNP_A-1651567 | | B, B | 112:24, 531:93 | 0.645 | 0.422 | 0.432 |
| | SNP_A-1709743 | | B, B | 116:26, 570:112 | 0.300 | 0.584 | 0.621 |
| | SNP_A-1758797 | | B, B | 116:24, 566:110 | 0.064 | 0.800 | 0.802 |
| | SNP_A-1691603 | | A, A | 95:43, 491:183 | 0.916 | 0.339 | 0.349 |
| | SNP_A-1754356 | | B, B | 116:26, 570:112 | 0.300 | 0.584 | 0.621 |
| | SNP_A-1753877 | | A, A | 86:56, 432:250 | 0.389 | 0.533 | 0.567 |
| 2 | Block 978-ABB | 0.046 | | 16.3:123.7, 22.0:660.0 | 18.638 | 0.0000158 | |
| | SNP_A-1737340 | | A, A | 131:7, 603:67 | 3.340 | 0.068 | 0.075 |
| | SNP_A-1737458 | | A, A | 116:24, 589:93 | 1.170 | 0.279 | 0.289 |
| | SNP_A-1737560 | | A, A | 91:47, 459:223 | 0.096 | 0.757 | 0.766 |
| 3 | Block 882-BAABB | 0.013 | | 7.0:135.0, 4.0:684.0 | 17.154 | 0.000034472 | |
| | SNP_A-1711934 | | B, B | 112:30, 557:131 | 0.328 | 0.567 | 0.561 |
| | SNP_A-1755635 | | A, A | 109:33, 535:153 | 0.068 | 0.795 | 0.825 |
| | SNP_A-1737814 | | B, B | 100:38, 525:151 | 1.738 | 0.187 | 0.186 |
| | SNP_A-1715417 | | B, B | 100:26, 517:133 | 0.002 | 0.965 | 1.000 |
| | SNP_A-1693406 | | A, A | 92:48, 497:185 | 2.932 | 0.087 | 0.099 |
| 5 | Block 1111-AABABBBBBB | 0.311 | | 64.0:76.0, 192.9:488.7 | 16.362 | 0.00005232 | |
| | SNP_A-1747985 | | B, B | 73:67, 481:201 | 17.868 | 0.000 | 0.000 |
| | SNP_A-1671292 | | B, B | 74:66, 479:203 | 15.932 | 0.000 | 0.000 |
| | SNP_A-1652471 | | B, B | 109:31, 452:230 | 7.190 | 0.007 | 0.007 |
| | SNP_A-1661474 | | A, B | 82:58, 374:302 | 8.991 | 0.003 | 0.003 |
| | SNP_A-1678546 | | B, B | 121:19, 533:149 | 4.893 | 0.027 | 0.029 |
| | SNP_A-1706814 | | B, B | 125:15, 578:98 | 1.391 | 0.238 | 0.283 |
| | SNP_A-1708155 | | B, B | 114:26, 508:174 | 3.040 | 0.081 | 0.084 |
| | SNP_A-1708263 | | A, A | 73:67, 477:205 | 16.620 | 0.000 | 0.000 |
| | SNP_A-1685724 | | B, B | 113:15, 497:117 | 3.898 | 0.048 | 0.056 |
| | SNP_A-1738519 | | B, B | 121:19, 535:147 | 4.593 | 0.032 | 0.037 |
| 4 | Block 160-BABA | 0.184 | | 43.1:98.9, 109.2:576.8 | 16.277 | 0.000054714 | |
| | SNP_A-1740315 | | B, A | 79:61, 370:304 | 5.967 | 0.015 | 0.016 |
| | SNP_A-1685351 | | A, A | 84:52, 331:325 | 5.774 | 0.016 | 0.018 |
| | SNP_A-1686635 | | B, B | 129:11, 627:59 | 0.083 | 0.774 | 0.869 |
| | SNP_A-1730571 | | A, A | 104:36, 477:203 | 0.963 | 0.326 | 0.359 |
| 1 | Block 119-AB | 0.294 | | 60.9:81.1, 178.1:503.9 | 16.01 | 0.000063012 | |
| | SNP_A-1754998 | | B, B | 77:59, 501:177 | 16.423 | 0.000 | 0.000 |
| | SNP_A-1745272 | | B, B | 116:26, 530:152 | 1.098 | 0.295 | 0.315 |
| 8 | Block 940-ABAB | 0.122 | | 31.2:108.8, 70.1:611.9 | 15.573 | 0.000079383 | |
| | SNP_A-1742854 | | A, A | 131:11, 604:84 | 2.313 | 0.128 | 0.148 |
| | SNP_A-1659634 | | A, A | 107:31, 602:68 | 16.141 | 0.000 | 0.000 |
| | SNP_A-1654329 | | A, A | 94:46, 441:235 | 0.187 | 0.666 | 0.697 |
| | SNP_A-1732459 | | B, B | 79:57, 347:309 | 1.222 | 0.269 | 0.299 |
| 15 | Block 352-AA | 0.127 | | 32.0:108.0, 73.1:608.9 | 15.376 | 0.00008811 | |
| | SNP_A-1739083 | | A, A | 129:13, 590:92 | 1.986 | 0.159 | 0.169 |
| | SNP_A-1704514 | | B, B | 103:37, 505:175 | 0.029 | 0.865 | 0.916 |
| | SNP_A-1704658 | | A, A | 132:8, 642:36 | 0.037 | 0.847 | 0.837 |
| | SNP_A-1706054 | | A, A | 104:36, 507:173 | 0.005 | 0.946 | 1.000 |
| | SNP_A-1658934 | | A, A | 71:69, 344:338 | 0.003 | 0.953 | 1.000 |
| 7 | Block 605-ABB | 0.245 | | 17.0:125.0, 188.0:494.0 | 15.292 | 0.00009209 | |
| | SNP_A-1702365 | | A, A | 95:25, 517:115 | 0.463 | 0.496 | 0.523 |
| | SNP_A-1677855 | | A, A | 122:18, 493:189 | 13.605 | 0.000 | 0.000 |
| | SNP_A-1672398 | | A, A | 125:17, 493:189 | 15.531 | 0.000 | 0.000 |
| 3 | Block 949-BBBB | 0.149 | | 36.2:103.8, 86.8:589.2 | 15.276 | 0.000092884 | |
| | SNP_A-1689062 | | A, A | 92:28, 549:65 | 14.741 | 0.000 | 0.000 |
| | SNP_A-1646498 | | B, B | 118:22, 582:100 | 0.102 | 0.750 | 0.794 |
| | SNP_A-1674729 | | A, A | 69:51, 434:178 | 8.399 | 0.004 | 0.005 |
| | SNP_A-1750459 | | A, A | 74:54, 444:172 | 10.198 | 0.001 | 0.002 |
| 6 | Block 431-BBB | 0.225 | | 50.1:91.9, 137.7:544.3 | 15.262 | 0.000093559 | |
| | SNP_A-1694932 | | A, A | 89:49, 536:134 | 15.707 | 0.000 | 0.000 |
| | SNP_A-1755968 | | B, B | 120:16, 565:81 | 0.062 | 0.804 | 0.887 |
| | SNP_A-1737300 | | B, B | 109:31, 492:178 | 1.184 | 0.277 | 0.290 |
| 2 | Block 1390-BBBA | 0.106 | | 2.0:138.0, 86.0:596.0 | 15.192 | 0.00009712 | |
| | SNP_A-1667363 | | B, B | 108:32, 545:133 | 0.757 | 0.384 | 0.418 |
| | SNP_A-1670809 | | A, A | 138:2, 594:86 | 15.252 | 0.000 | 0.000 |
| | SNP_A-1713136 | | A, A | 101:39, 440:242 | 3.003 | 0.083 | 0.096 |
| | SNP_A-1687698 | | B, B | 106:36, 447:235 | 4.415 | 0.036 | 0.039 |
| 1 | Block 1192-AA | 0.33 | | 66.0:76.0, 203.0:479.0 | 14.931 | 0.0001 | |
| | SNP_A-1643025 | | A, A | 118:24, 544:138 | 0.827 | 0.363 | 0.417 |
| | SNP_A-1646176 | | A, — | 90:52, 341:341 | 8.435 | 0.004 | 0.004 |
| 5 | Block 498-BBB | 0.159 | | 37.0:103.0, 95.0:587.0 | 13.462 | 0.0002 | |
| | SNP_A-1703319 | | B, B | 104:36, 501:181 | 0.041 | 0.840 | 0.916 |
| | SNP_A-1720661 | | B, B | 104:36, 501:181 | 0.041 | 0.840 | 0.916 |
| | SNP_A-1712558 | | A, A | 103:37, 587:95 | 13.462 | 0.000 | 0.001 |
| 7 | Block 558-AA | 0.496 | | 91.2:50.8, 319.0:363.0 | 14.301 | 0.0002 | |
| | SNP_A-1706639 | | A, A | 134:8, 619:63 | 1.938 | 0.164 | 0.190 |
| | SNP_A-1657336 | | A, B | 89:49, 363:319 | 14.414 | 0.000 | 0.000 |

Combined results of genetic associations observed by single-point and haplotype assessments, ranked by chi-square outcome, are summarized in TABLE 4 below.

TABLE 4

| | | Chi | |
|---|---|---|---|
| SNP or Block | Chromosome | Square | p-value |
| 7-Block 357-AABBBBBB | 7 | 19.771 | 8.73E−06 |
| 2-Block 978-ABB | 2 | 18.638 | 1.58E−05 |
| SNP_A-1747985 | 5 | 17.868 | 2.37E−05 |
| SNP_A-1728960 | 1 | 17.434 | 2.97E−05 |
| SNP_A-1744334 | 7 | 17.365 | 3.08E−05 |
| 3-Block 882-BAABB | 3 | 17.154 | 3.45E−05 |
| SNP_A-1708263 | 5 | 16.620 | 4.57E−05 |
| SNP_A-1653812 | 18 | 16.537 | 4.77E−05 |
| SNP_A-1754998 | 1 | 16.423 | 5.07E−05 |
| SNP_A-1654099 | 5 | 16.398 | 5.13E−05 |
| 5-Block 1111-AABABBBBBB | 5 | 16.362 | 5.23E−05 |
| 4-Block 160-BABA | 4 | 16.277 | 5.47E−05 |
| SNP_A-1659634 | 8 | 16.141 | 5.88E−05 |
| 1-Block 119-AB | 1 | 16.010 | 6.30E−05 |
| SNP_A-1671292 | 5 | 15.932 | 6.56E−05 |
| SNP_A-1694932 | 6 | 15.707 | 7.39E−05 |
| SNP_A-1684457 | 8 | 15.702 | 7.42E−05 |
| SNP_A-1750208 | 2 | 15.614 | 7.77E−05 |
| 8-Block 940-ABAB | 8 | 15.573 | 7.94E−05 |
| SNP_A-1672398 | 7 | 15.531 | 8.12E−05 |
| SNP_A-1651422 | 1 | 15.506 | 8.22E−05 |
| 15-Block 352-AA | 15 | 15.376 | 8.81E−05 |
| 7-Block 605-ABB | 7 | 15.292 | 9.21E−05 |
| 3-Block 949-BBBB | 3 | 15.276 | 9.29E−05 |
| 6-Block 431-BBB | 6 | 15.262 | 9.36E−05 |
| SNP_A-1670809 | 2 | 15.252 | 9.41E−05 |
| 2-Block 1390-BBBA | 2 | 15.192 | 9.71E−05 |
| 1-Block 1192-AA | 1 | 14.931 | 0.0001 |
| SNP_A-1690321 | 15 | 14.461 | 0.0001 |
| SNP_A-1657336 | 7 | 14.414 | 0.0001 |
| SNP_A-1689062 | 3 | 14.741 | 0.0001 |
| SNP_A-1686758 | 15 | 14.461 | 0.0001 |
| SNP_A-1663754 | 22 | 14.555 | 0.0001 |
| SNP_A-1713325 | 6 | 14.917 | 0.0001 |
| 5-Block 498-BBB | 5 | 13.462 | 0.0002 |
| 7-Block 558-AA | 7 | 14.301 | 0.0002 |
| 10-Block 201-AABBBB | 10 | 13.645 | 0.0002 |
| 11-Block 351-BBB | 11 | 13.497 | 0.0002 |
| SNP_A-1689777 | 17 | 13.834 | 0.0002 |
| SNP_A-1697299 | 3 | 14.367 | 0.0002 |
| SNP_A-1646220 | 13 | 14.045 | 0.0002 |
| SNP_A-1649117 | 3 | 13.873 | 0.0002 |
| SNP_A-1677855 | 7 | 13.605 | 0.0002 |
| SNP_A-1642608 | 6 | 13.428 | 0.0002 |
| SNP_A-1744891 | 9 | 13.950 | 0.0002 |
| SNP_A-1712558 | 5 | 13.462 | 0.0002 |
| SNP_A-1672476 | 7 | 13.486 | 0.0002 |
| SNP_A-1648183 | 11 | 13.509 | 0.0002 |
| SNP_A-1666270 | 15 | 14.360 | 0.0002 |
| SNP_A-1663412 | 4 | 14.111 | 0.0002 |
| SNP_A-1689621 | 8 | 14.363 | 0.0002 |
| SNP_A-1674524 | 18 | 14.248 | 0.0002 |
| 3-Block 631-BB | 3 | 12.935 | 0.0003 |
| 6-Block 213-BABB | 6 | 13.227 | 0.0003 |
| SNP_A-1648505 | 11 | 13.002 | 0.0003 |
| SNP_A-1674414 | 18 | 12.861 | 0.0003 |
| SNP_A-1752407 | 2 | 12.797 | 0.0003 |
| SNP_A-1758088 | 6 | 12.949 | 0.0003 |
| SNP_A-1651632 | 11 | 13.171 | 0.0003 |
| SNP_A-1652367 | 18 | 13.405 | 0.0003 |
| SNP_A-1752416 | 3 | 13.202 | 0.0003 |
| SNP_A-1714418 | 8 | 13.048 | 0.0003 |
| SNP_A-1681449 | 3 | 12.913 | 0.0003 |
| SNP_A-1657052 | 2 | 13.116 | 0.0003 |
| SNP_A-1748758 | 3 | 13.371 | 0.0003 |
| SNP_A-1668805 | 2 | 12.949 | 0.0003 |
| 1-Block 156-BAAAA | 1 | 12.734 | 0.0004 |
| 2-Block 82-ABA | 2 | 12.685 | 0.0004 |
| 2-Block 1516-ABBB | 2 | 12.488 | 0.0004 |
| 3-Block 631-AA | 3 | 12.659 | 0.0004 |
| 6-Block 982-AAABABAABB | 6 | 12.507 | 0.0004 |
| 8-Block 417-AA | 8 | 12.657 | 0.0004 |
| 8-Block 682-BAAB | 8 | 12.553 | 0.0004 |
| 10-Block 98-BA | 10 | 12.480 | 0.0004 |
| 13-Block 183-ABBAAAAABAAB | 13 | 12.483 | 0.0004 |
| 17-Block 423-ABBB | 17 | 12.716 | 0.0004 |
| SNP_A-1681731 | 3 | 12.659 | 0.0004 |
| SNP_A-1658214 | 5 | 12.611 | 0.0004 |
| SNP_A-1673435 | 13 | 12.674 | 0.0004 |
| SNP_A-1713453 | 12 | 12.752 | 0.0004 |
| SNP_A-1747301 | 16 | 12.371 | 0.0004 |
| SNP_A-1746683 | 22 | 12.508 | 0.0004 |
| 6-Block 62-AA | 6 | 12.289 | 0.0005 |
| 13-Block 55-BA | 13 | 11.995 | 0.0005 |
| 13-Block 182-BBB | 13 | 11.992 | 0.0005 |
| 17-Block 423-BAAA | 17 | 12.255 | 0.0005 |
| SNP_A-1755442 | 18 | 12.024 | 0.0005 |
| SNP_A-1729831 | 16 | 12.137 | 0.0005 |
| SNP_A-1725694 | 1 | 12.209 | 0.0005 |
| SNP_A-1722723 | 11 | 11.948 | 0.0005 |
| SNP_A-1737843 | 5 | 12.016 | 0.0005 |
| SNP_A-1690009 | 18 | 12.024 | 0.0005 |
| SNP_A-1709339 | 14 | 11.965 | 0.0005 |
| SNP_A-1703326 | 6 | 11.997 | 0.0005 |
| SNP_A-1644949 | 14 | 12.139 | 0.0005 |
| SNP_A-1721181 | 3 | 12.286 | 0.0005 |
| SNP_A-1713954 | 8 | 12.306 | 0.0005 |
| SNP_A-1753986 | 3 | 12.260 | 0.0005 |
| SNP_A-1714568 | 13 | 12.262 | 0.0005 |
| SNP_A-1728025 | 10 | 12.300 | 0.0005 |

As shown in Table 5, top genetic associations involved SNPs in or near GRIA1 (Glutamate receptor, ionotropic, AMPA 1), AK5 (Adenylate kinase 5), SLC1A3 (Solute carrier family 1 (glial high affinity glutamate transporter), member 3), BAI3 (brain-specific angiogenesis inhibitor 3) and CACNA2D1 (calcium channel, voltage-dependent, alpha 2/delta subunit 1).

TABLE 5
GENES ASSOCIATED WITH MCI TO AD CONVERSION

| | Known neurological | |
|---|---|---|
| Genes | association? | Comments |
| GRIA1 | X | Glutamate receptor |
| AK5 | | Brain specific |
| SLC1A3 | X | Glutamate transporter |
| BAB | | Brain specific |
| CACNA2D1 | | Calcium channel |

Single Marker Association. Assessment of single genetic marker association for conversion of MCI to AD was performed on 109,768 markers. Allele frequencies were compared between 72 cases and 347 controls. The top 25 single point gene mutations (biomarkers of the invention) identified as associated with conversion of MCI to AD are summarized in TABLE 6.

TABLE 6
Single Point Gene Mutations Identified as Associated with Conversion of MCI to AD

| | | | dbSNP | Major | | | | Fisher's | |
|---|---|---|---|---|---|---|---|---|---|
| Name | Chrome | Position | RS ID | alleles | Case:Control | Chi square | p-value | p-value | Gene |
| SNP_A-1670809 | 2 | 214935064 | rs10490502 | A, A | 138:2, 594:86 | 15.25 | 9.41E−05 | 8.55E−06 | SPAG16 |
| SNP_A-1750208 | 2 | 124721329 | rs1170585 | A, A | 115:5, 507:117 | 15.61 | 7.77E−05 | 2.03E−05 | CNTNAP5 |
| SNP_A-1747985 | 5 | 153057231 | rs4415128 | B, B | 73:67, 481:201 | 17.87 | 2.37E−05 | 4.34E−05 | GRIA1 |
| SNP_A-1672398 | 7 | 86116564 | rs17126 | A, A | 125:17, 493:189 | 15.53 | 8.12E−05 | 4.42E−05 | — |
| SNP_A-1654099 | 5 | 36703336 | rs10491374 | B, A | 93:47, 353:321 | 16.40 | 5.13E−05 | 5.88E−05 | SLC1A3 |
| SNP_A-1653812 | 18 | 60211031 | rs1833486 | A, B | 85:51, 367:281 | 16.54 | 4.77E−05 | 6.46E−05 | C18orf20 |
| SNP_A-1708263 | 5 | 153177889 | rs2964013 | A, A | 73:67, 477:205 | 16.62 | 4.57E−05 | 7.29E−05 | GRIA1 |
| | | | | | | | | | C5orf3 |
| SNP_A-1646220 | 13 | 41916177 | rs720824 | A, A | 127:13, 522:160 | 14.05 | 2.00E−04 | 9.28E−05 | TNFSF11 |
| SNP_A-1671292 | 5 | 153067976 | rs7735784 | B, B | 74:66, 479:203 | 15.93 | 6.56E−05 | 1.05E−04 | GRIA1 |
| SNP_A-1754998 | 1 | 43906558 | rs7516647 | B, B | 77:59, 501:177 | 16.42 | 5.07E−05 | 1.12E−04 | ST3GAL3 |
| SNP_A-1744334 | 7 | 16181205 | rs1921840 | A, A | 93:35, 539:79 | 17.37 | 3.08E−05 | 1.20E−04 | — |
| SNP_A-1694932 | 6 | 66726185 | rs2061578 | A, A | 89:49, 536:134 | 15.71 | 7.39E−05 | 1.36E−04 | BAI3 |
| SNP_A-1677855 | 7 | 86115205 | rs6465088 | A, A | 122:18, 493:189 | 13.61 | 2.00E−04 | 1.61E−04 | — |
| SNP_A-1659634 | 8 | 129710034 | rs10505528 | A, A | 107:31, 602:68 | 16.14 | 5.88E−05 | 1.71E−04 | MGC27434 |
| SNP_A-1657336 | 7 | 81502544 | rs929351 | A, B | 89:49, 363:319 | 14.41 | 1.00E−04 | 1.75E−04 | CACNA2D1 |
| | | | | | | | | | LRRC19 |
| SNP_A-1742608 | 9 | 27057529 | rs10511798 | B, B | 136:0, 627:47 | 10.07 | 1.50E−03 | 1.94E−04 | TEK CCDC2 |
| SNP_A-1681449 | 3 | 103365250 | rs9290621 | A, A | 129:13, 532:154 | 12.91 | 3.00E−04 | 2.04E−04 | NFKBIZ |
| | | | | | | | | | LOC131368 |
| SNP_A-1674524 | 18 | 60165056 | rs4306624 | A, B | 80:56, 394:276 | 14.25 | 2.00E−04 | 2.06E−04 | C18orf20 |
| SNP_A-1689621 | 8 | 56479295 | rs2622542 | B, B | 66:58, 427:177 | 14.36 | 2.00E−04 | 2.15E−04 | — |
| SNP_A-1728960 | 1 | 77566211 | rs10518551 | A, A | 121:19, 652:30 | 17.43 | 2.97E−05 | 2.26E−04 | AK5 |
| SNP_A-1666270 | 15 | 31572775 | rs1369308 | A, A | 70:66, 461:213 | 14.36 | 2.00E−04 | 2.33E−04 | — |
| SNP_A-1738563 | 18 | 19749481 | rs1541836 | A, A | 137:5, 587:91 | 11.13 | 8.00E−04 | 2.98E−04 | LAMA3 |
| SNP_A-1748758 | 3 | 109243490 | rs696365 | A, B | 86:56, 387:301 | 13.37 | 3.00E−04 | 2.99E−04 | CD47 BBX |
| SNP_A-1652367 | 18 | 35487281 | rs10502726 | B, A | 76:38, 294:270 | 13.41 | 3.00E−04 | 3.00E−04 | PIK3C3 |
| SNP_A-1752416 | 3 | 109276399 | rs3804640 | A, B | 86:56, 383:299 | 13.20 | 3.00E−04 | 3.04E−04 | CD47 |

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

We claim:

1. Use of rivastigmine (Exelon) in the manufacture of a medicament for the treatment of Alzheimer's Disease with a reduced toxicity or increased effect in a selected subject population, wherein the subject population is selected on the basis of the presence of at least one gene mutation selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; MUT-25.

2. A method for treating Alzheimer's Disease in a subject, comprising the steps of:
(a) obtaining the genotype or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11, wherein the genotype and/or haplotype is indicative of a propensity for having Alzheimer's Disease; and
(b) administering an anti-Alzheimer's Disease therapy to the subject.

3. The method of claim 2, wherein the anti-Alzheimer's Disease therapy is selected from the group consisting of: tacrine; donepezil; rivastigmine; galantamine; and memantine.

4. The method of claim 2, wherein the genotype is heterozygous, with at least one of the alleles containing a genetic polymorphism and/or mutation selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; MUT-25.

5. The method of claim 2, wherein the genotype is homozygous, with at least one of the alleles containing a mutation and/or polymorphism of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25.

6. The method of claim 2, wherein the anti-Alzheimer's Disease therapy is the administration of a therapeutically effective amount of an agent which increases or decreases the level of expression of a gene comprising a genetic mutation or polymorphism selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25.

7. A method for identifying a subject with a disorder for which an Alzheimer's Disease-associated mutation identified in TABLE 1 is predictive, comprising the steps of:
(a) obtaining the genotype or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11;
(b) assessing the genotype and/or haplotype to determine the presence of a mutation or polymorphism selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25, wherein the presence of the mutation or polymorphism is indicative of a propensity of the subject to have a disorder for which the Alzheimer's Disease-associated mutation identified in TABLE 1 is predictive; and
(c) identifying the subject as having a propensity for having a disorder for which an Alzheimer's Disease-associated mutation identified in TABLE 1 is predictive.

8. The method of claim 7, wherein the disorder for which the Alzheimer's Disease-associated mutations identified in TABLE 1 are predictive is Alzheimer's Disease

9. A method for determining, prior to initiation of treatment, whether a subject should be included in a study of a therapeutic or study agent; comprising:
(a) interrogating the genotype and/or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11;
(b) then:
including the subject in the study if the genotype is indicative of a propensity to Alzheimer's Disease by the subject;
(ii) excluding the subject from the study if the genotype is not indicative of a propensity to Alzheimer's Disease by the subject; or
(iii) both (i) and (ii).

10. A method for determining the responsiveness of a subject with a disorder to treatment, comprising the steps of:
(a) obtaining the genotype or haplotype of the subject at a genetic locus or loci of at least one gene selected from the group consisting of: AK5; BAI3; BBX; C18orf20; C5orf3; CACNA2D1; CCDC2; CD47; CNTNAP5; GRIM; LAMA3; LOC131368; LRRC19; MGC27434; NFKBIZ; PIK3C3; SLC1A3; SPAG16; ST3GAL3; TEK; and TNFSF11;
(b) assessing the genotype and/or haplotype to determine the presence of a mutation or polymorphism selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25, wherein the presence of the mutation or polymorphism is indicative of a subject that is responsive to treatment of the disorder; and
(c) identifying the subject as responsive to treatment of the disorder.

11. A method for determining, prior to treatment, a subject that will develop toxicity when treated with a compound; comprising:
(a) obtaining the genotype or haplotype of the subject at a genetic locus or loci of at least one gene selected from the group consisting of: AK5; BAI3; BBX; C18orf20; C5orf3; CACNA2D1; CCDC2; CD47; CNTNAP5; GRIA1; LAMA3; LOC131368; LRRC19; MGC27434; NFKBIZ; PIK3C3; SLC1A3; SPAG16; ST3GAL3; TEK; and TNFSF11;
(b) assessing the genotype and/or haplotype to determine the presence of a mutation or polymorphism selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25, wherein the presence of the mutation or polymorphism is indicative of a subject that will develop toxicity when treated with the compound; and
(c) identifying the subject as a subject that will develop toxicity when treated with the compound.

12. A method for monitoring the progression or development of toxicity in a subject being treated with a compound, the method comprising:
(a) providing a first test biological sample from the subject;
(b) providing a second test biological sample from the subject which is later in time than the first test biological sample;
(c) contacting the test biological samples with a reagent for detecting a polynucleotide or polypeptide encoded by a gene having a sequence comprising a mutation selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25;
(d) determining the level of expression of the polypeptide or polynucleotide in the test biological samples; and
(e) comparing the level of the polynucleotide or polypeptide level in the first test biological sample with the level of the polynucleotide or polypeptide in the second test biological sample, wherein an increase or a decrease in the level of polynucleotide or polypeptide in the second test biological sample relative to the level of the polynucleotide or polypeptide in the first test biological sample indicates the progression or development of toxicity in the subject being treated with the compound.

13. A method for determination of when treatment with a compound should be discontinued in a subject at risk of having, toxicity during or after treatment with the compound, comprising the steps of:
(a) providing a test biological sample;
(b) contacting the test biological sample with a reagent for detecting a polynucleotide or polypeptide encoded by a gene having a sequence comprising a mutation selected from the group consisting of: MUT-1; MUT-2; MUT-3; MUT-4; MUT-5; MUT-6; MUT-7; MUT-8; MUT-9; MUT-10; MUT-11; MUT-12; MUT-13; MUT-14; MUT-15; MUT-16; MUT-17; MUT-18; MUT-19; MUT-20; MUT-21; MUT-22; MUT-23; MUT-24; and MUT-25,
(c) determining the level of expression of the polypeptide or polynucleotide in the test biological sample; and
(d) comparing the level of the polynucleotide or polypeptide level in the test biological sample with the level of the polynucleotide or polypeptide in a standard reference sample, wherein similarity between the level of polynucleotide or polypeptide and the level of the polynucleotide or polypeptide in the standard reference sample is indicative of the development of toxicity in the subject and determines that the compound should be discontinued.
